(19) United States
(12) Patent Application Publication
(10) Pub. No.: US 2009/0143456 A1
(43) Pub. Date: Jun. 4, 2009

(54) Polyamine Analogs as Modulators of Cell Migration and Cell Motility

(76) Inventors: Laurence J. Marton, Palo Alto, CA (US); Michael E. Berens, Gillbert, AZ (US); Benjamin Frydman, Madison, WI (US); Linda CLifford, Madison, WI (US)

Correspondence Address: MORRISON & FOERSTER LLP, 755 PAGE MILL RD, PALO ALTO, CA, 94304-1018, US

(21) Appl. No.: 11/884,165

(22) PCT Filed: Feb. 13, 2006
(86) PCT No.: PCT/US06/05041

§ 371 Date: Oct. 22, 2008

Related U.S. Application Data
(60) Provisional application No. 60/652,193, filed on Feb. 11, 2005.

(51) Int. Cl. A61K 31/409; A61K 31/132; A61P 43/00

(52) U.S. Cl. 514/410; 514/659; 514/646

(57) ABSTRACT

This disclosure relates to methods of inhibiting cell motility or cell migration, and of treating diseases involving cell migration or cell motility, using polyamine analogs, such as conformationally restricted polyamine analogs. The diseases to be treated include immune disorders, inflammatory conditions, infection, abnormal immune responses, undesired angiogenesis, tumor cell metastasis or invasion, atherosclerosis, vascular graft occlusion, transplant rejection, other complications of transplants, glomerulonephritis, arthritis, inflammatory responses subsequent to stroke or ischemia, and asthma.

### CROSS REFERENCE TO RELATED APPLICATIONS

This application claims the benefit of U.S. Provisional Application Ser. No. 60/652,193, filed Feb. 11, 2005, which is hereby incorporated herein by reference in it's entirety.

### STATEMENT REGARDING FEDERALLY SPONSORED RESEARCH OR DEVELOPMENT

Not applicable.

### TECHNICAL FIELD

This application relates to methods of using polyamine analogs, particularly conformationally restricted polyamine analogs, to affect cell motility and migration, and to treat diseases, such as inflammation, by modulating cell motility and cell migration.

### BACKGROUND

Cell migration and cell motility is a complex phenomenon, involving (1) extension of the leading edge of the cell; (2) adhesion to matrix contacts; (3) contraction of the cytoplasm; (4) release from contact sites; and (5) recycling of membrane receptors from the rear to the front of the cell (Sheetz et al., Biochem Soc Symp. 65:233 (1999)). Cell migration is a significant component in many normal and pathological biological events, such as development, angiogenesis, wound healing, the immune response and the metastasis and invasion of tumor cells. Accordingly, compositions and methods that affect cell migration provide potential avenues of treating diseases involving cell motility or cell migration. Examples of therapeutic intervention via modulation of cell motility or cell migration are described in International Patent Application No. WO 01/74853 and U.S. Patent Application No. 2005/0014675.

Polyamines are a ubiquitous class of compounds found in both prokaryotic and eukaryotic cells. Polyamines are involved in a wide variety of physiological processes. McCormack and Johnson (J. Physiol. Pharmacol. 52:327 (2001)) discussed specific processes involving polyamines that may affect cell migration.

Conformationally-restricted polyamine analogs and methods of synthesizing such analogs have been disclosed ill U.S. Pat. Nos. 5,889,061, 6,392,098, and 6,794,545, United States Patent Application Publication Nos. 2003/0072715, 2003/0195377, and International Patent Applications WO 98/17624, WO 00/66587, WO 02/10142, and WO 03/050072. These compounds have been shown to have anti-cancer effects in vitro or in vivo.

The instant application relates to the use of polyamines and polyamine analogs, in particular conformationally-restricted polyamine analogs, for modulation of cell motility and cell migration, and treatment of diseases where cell motility and cell migration plays a role in the disease.

### DISCLOSURE OF THE INVENTION

In one embodiment, the invention relates to the use of polyamine analogs to modulate cell migration or cell motility. In another embodiment, the invention relates to the use of polyamine analogs to treat diseases involving cell migration or cell motility. In another embodiment, the polyamine analogs are conformationally restricted.

In one embodiment, the conformationally restricted polyamine analog is selected from among compounds of the formula:

E-N-H-B-A-B-NH-B-A-B-NH-B-A-B-NH-B-A-B-NH-E

where A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of: a single bond, C₁-C₆ alkyl, and C₂-C₆ alkenyl; and E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. Specific embodiments of compounds of this type include

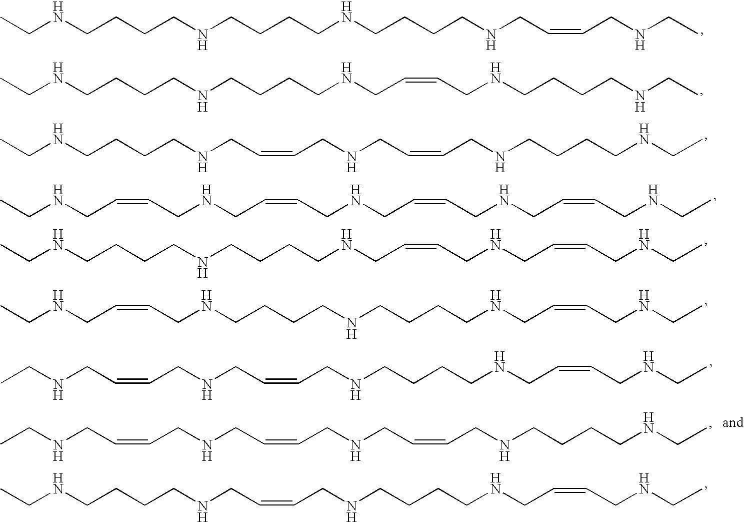

and all salts, hydrates, solvates, and stereoisomers thereof.

In another embodiment, the conformationally restricted polyamine analog is selected from among the group of compounds of the formula:

E-NH-B-A-B-NH-B-A-B-NH-B-A-B-NH(-B-A-B-NH)ₓ-E

wherein A is independently selected from the group consisting of C₁-C₆ all-yl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of H, C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; and x is an integer from 2 to 16; with the proviso that either at least one A moiety is selected from the group consisting of C₂-C₆ alkenyl, C₂-C₆ alkynyl, C₃-C₆ cycloalkyl, C₃-C₆ cycloaryl, and C₃-C₆ cycloalkenyl, or at least one B moiety is selected from the group consisting of C₂-C₆ alkenyl; and all salts, hydrates, solvates, and stereoisomers thereof. Specific embodiments of compounds of this type include

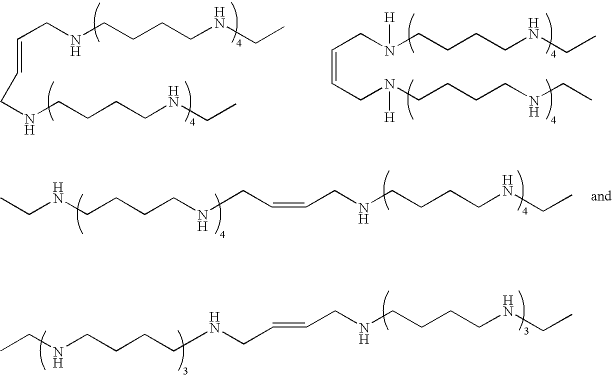

and all salts, hydrates, solvates, and stereoisomers thereof.

h n another embodiment, the conformationally restricted polyamine analog is selected from among the group of compounds of the formula

E-NH-B-A-B-NH-B-A-B-NH-B-A-B-NH(-B-A-B-NH)ₓ-E

wherein A is independently selected from the group consisting of C₁-C₆ alkyl, C₂-C₆ alkenyl, C₂-C₆ alkynyl, C₃-C₆ cycloalkyl, C₃-C₆ cycloaryl, and C₃-C₆ cycloalkenyl; B is independently selected from the group consisting of a single bond, C₁-C₆ alkyl, and C₂-C₆ alkenyl; E is independently selected from the group consisting of C₁-C₆ alkyl, C₁-C₆ alkanol, C₃-C₆ cycloalkanol, and C₃-C₆ hydroxyaryl, with the proviso that at least one E moiety be selected from the group consisting of C₁-C₆ alkanol, C₃-C₆ cycloalkanol, and C₃-C₆ hydroxyaryl; and x is an integer from 0 to 16; and all salts, hydrates, solvates, and stereoisomers thereof. Specific embodiments of compounds of this type include

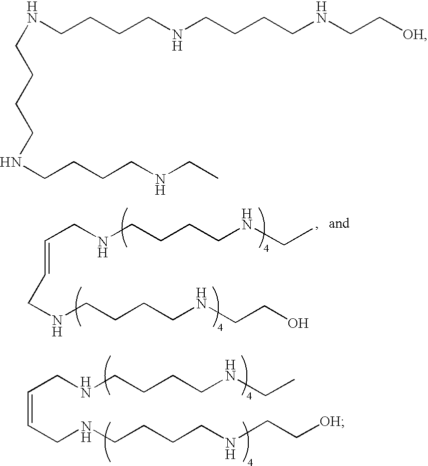

and all salts, hydrates, solvates, and stereoisomers thereof.

In another embodiment, the conformationally restricted polyamine analog is selected from among the group of compounds of the formula

E-NH-D-NH-B-A-B-NH-D-NH-E

wherein A is selected from the group consisting of C₂-C₆ alkene and C₃-C₆ cycloalkyl, cycloalkenyl, and cycloaryl; B is independently selected from the group consisting of a single bond and C₁-C₆ alkyl and alkenyl; D is independently selected from the group consisting of C₁-C₆ alkyl and alkenyl, and C₃-C₆ cycloalkyl, cycloalkenyl, and cycloaryl; E is independently selected from the group consisting of H, C₁-C₆ alkyl and alkenyl; and all salts, hydrates, solvates, and stereoisomers thereof. A specific embodiment of compounds of this type includes

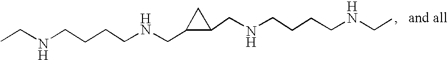

salts, hydrates, solvates, and stereoisomers thereof.

In another embodiment, the conformationally restricted polyamine analog is selected from macrocyclic polyamines of the formula:

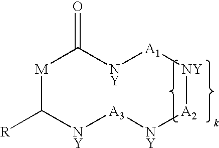

where A₁, each A₂ (if present), and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In additional embodiments, the Y group is —H or —CH₃. In another embodiment, A₁, each A₂ (if present), and A₃ are independently selected from C₂-C₄ alkyl. In yet another embodiment, M is —CH₂—.

In another embodiment, the conformationally restricted polyamine analog is selected from macrocyclic polyamine analogs of the formula

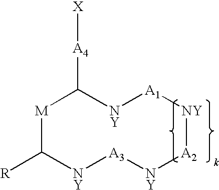

where A₁, each A₂ (if present), and A₃ are independently selected from C₁-C₈ alkyl; where A₄ is selected from C₁-C₈ alkyl or a nonentity; where X is selected from —H, -Z, —CN, —NH₂, —C(═O)—C₁-C₈ alkyl, or —NHZ, with the proviso that when A₄ is a nonentity, X is —H, —C(═O)—C₁-C₉ alkyl, or -Z; where Z is selected from the group consisting of an amino protecting group, an amino capping group, an amino acid, and a peptide; where each Y is independently selected from H or C₁-C₄ alkyl; where M is selected from C₁-C₄ alkyl; where k is 0, 1, 2, or 3; and where R is selected from C₁-C₃₂ alkyl; and all salts, hydrates, solvates, and stereoisomers thereof. In certain embodiments, A₄ is a nonentity. In other embodiments, X is -Z, and -Z is —H. In other embodiments, Y is —CH₃. In other embodiments, M is —CH₂—. In still further embodiments, k is 1. In further embodiments, A₁ and A₃ are —CH₂CH₂CH₂—. In still further embodiments, —CH₂CH₂CH₂CH₂—. In still further embodiments, R is —C₁₃H₂₇. In yet further embodiments, one or more of the specific limitations on A₄, X, Z, Y, M, k, A₁, A₃, and R are combined.

In further embodiments of these compounds, A₄ is C₁-C₈ alkyl, X is —NHZ, and Z is selected from one of the 20 genetically encoded amino acids (alanine, cysteine, aspartic acid, glutamic acid, phenylalanine, glycine, histidine, isoleucine, lysine, methionine, asparagine, proline, glutamine, arginine, serine, threonine, valine, tryptophan, tyrosine), a peptide of the formula acetyl-SKLQL-, a peptide of the formula acetyl-SKLQ-β-alanine-, or a peptide of the formula acetyl-SKLQ-. In these cases, where Z is an amino acid or peptide, the therapeutic agent to be used is a polyamine-amino acid conjugate or polyamine-peptide conjugate.

In another embodiment, the polyamine analog is selected from the group consisting of:

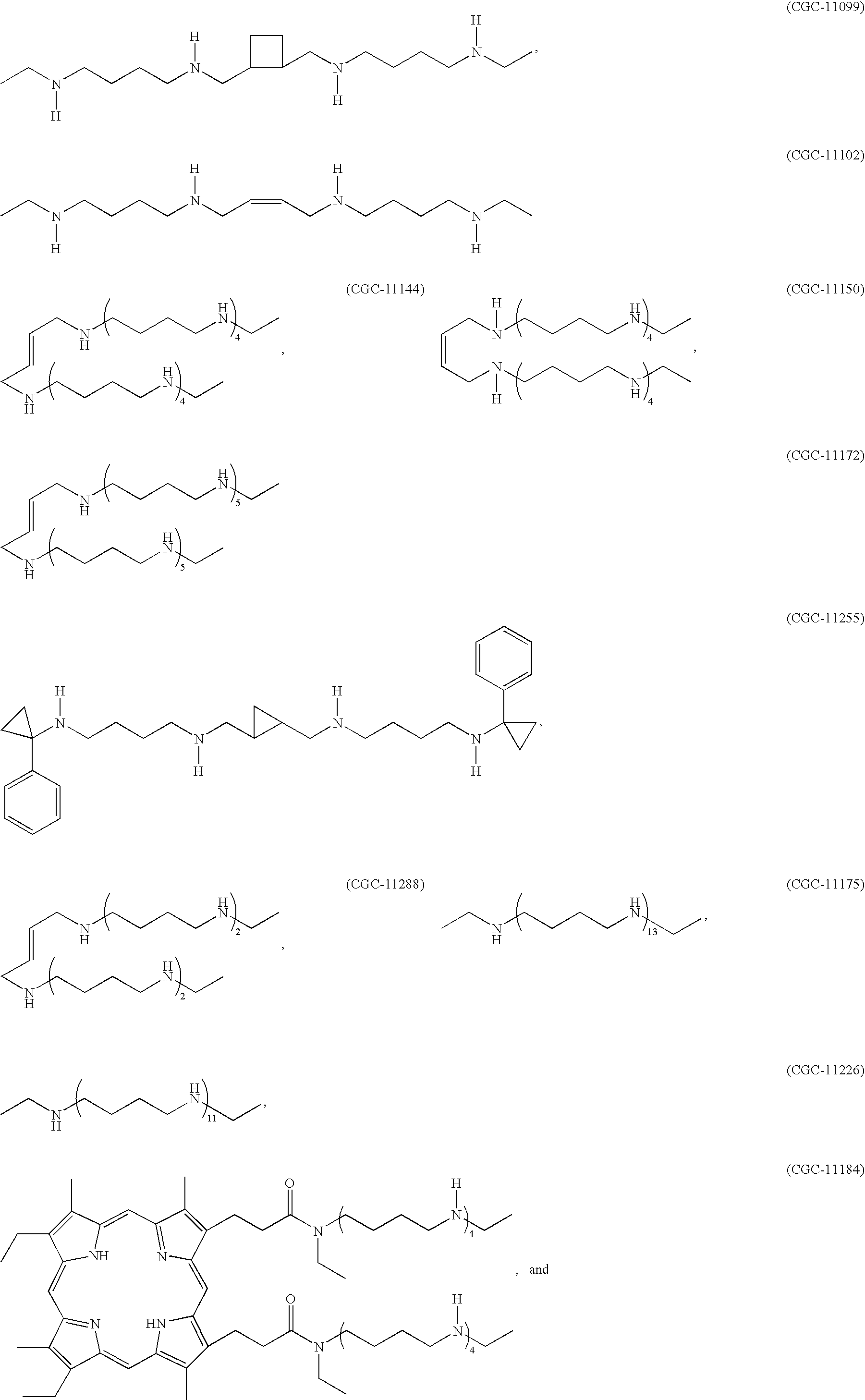

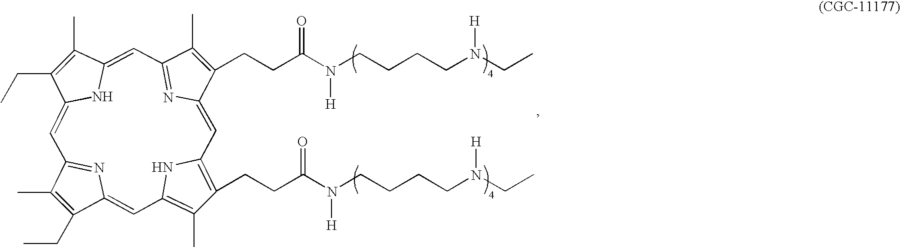

and all salts, hydrates, solvates, and stereoisomers thereof.

In another embodiment, the invention embraces a method of treating diseases involving cell migration or cell motility, comprising administering one or more polyamine analogs to a subject with a disease involving cell migration or cell motility in an amount sufficient to have a therapeutic effect on the disease involving cell migration or cell motility. Preferably, the polyamine analog is a conformationally restricted polyamine analog. The method embraces administration of the polyamine analog or conformationally restricted polyamine analog in an amount sufficient to reduce cell motility or cell migration. The invention also embraces administration of the polyamine analog or conformationally restricted polyamine analog in an amount sufficient to treat the disease involving cell migration or cell motility.

In another embodiment, the polyamine analog or conformationally restricted polyamine analog is present in pharmaceutically acceptable formulations, which comprise one or more polyamine analogs and pharmaceutically acceptable excipients. In another embodiment, the polyamine analog or conformationally restricted polyamine analog is present in a human unit dosage formulation.

### BRIEF DESCRIPTION OF THE DRAWINGS

FIG. 1 depicts the effects of compound SL-11047 (CGC-11047) on cell motility.

FIG. 2 depicts the effects of compound SL-11061 (CGC-11061) on cell motility.

FIG. 3 depicts the effects of compound SL-11090 (CGC-11090) on cell motility.

FIG. 4 depicts the effects of compound SL-11091 (CGC-11091) on cell motility.

FIG. 5 depicts the effects of compound SL-11099 (CGC-11099) on cell motility.

FIG. 6 depicts the effects of compound SL-11101 (CGC-11101) on cell motility.

FIG. 7 depicts the effects of compound SL-11102 (CGC-11102) on cell motility.

FIG. 8 depicts the effects of compound SL-11103 (CGC-11103) on cell motility.

FIG. 9 depicts the effects of compound SL-11122 (CGC-11122) on cell motility.

FIG. 10 depicts the effects of compound SL-11130 (CGC-11130) on cell motility.

FIG. 11 depicts the effects of compound SL-11144 (CGC-11144) on cell motility.

FIG. 12 depicts the effects of compound SL-11150 (CGC-11150) on cell motility.

FIG. 13 depicts the effects of compound SL-11157 (CGC-11157) on cell motility.

FIG. 14 depicts the effects of compound SL-11158 (CGC-11158) on cell motility.

FIG. 15 depicts the effects of compound SL-11159 (CGC-11159) on cell motility.

FIG. 16 depicts the effects of compound SL-11172 (CGC-11172) on cell motility.

FIG. 17 depicts the effects of compound SL-11175 (CGC-11175) on cell motility.

FIG. 18 depicts the effects of compound SL-11207 (CGC-11207) on cell motility.

FIG. 19 depicts the effects of compound SL-11226 (CGC-11226) on cell motility.

FIG. 20 depicts the effects of compound SL-11231 (CGC-11231) on cell motility.

FIG. 21 depicts the effects of compound SL-11251 (CGC-11251) on cell motility.

FIG. 22 depicts the effects of compound SL-11253 (CGC-11253) on cell motility.

FIG. 23 depicts the effects of compound SL-11255 (CGC-11255) on cell motility.

FIG. 24 depicts the effects of compound SL-11262 (CGC-11262) on cell motility.

FIG. 25 depicts the effects of compound SL-11287 (CGC-11287) on cell motility.

FIG. 26 depicts the effects of compound SL-11288 (CGC-11288) on cell motility.

FIG. 27 depicts the effects of compound SL-11215 (CGC-11215) on cell motility.

FIG. 28 depicts the effects of compound SL-11296 (CGC-11296) on cell motility.

FIG. 29 depicts the effects of compound SL-11295 (CGC-11295) on cell motility.

FIG. 30 depicts the effects of compound SL-11218 (CGC-11218) on cell motility.

FIG. 31 depicts the effects of compound SL-11293 (CGC-11293) on cell motility.

FIG. 32 depicts the effects of compound SL-11259 (CGC-11259) on cell motility.

FIG. 33 depicts the effects of compound SL-11286 (CGC-11286) on cell motility.

FIG. 34 depicts the effects of compound SL-11160 (CGC-11160) on cell motility.

FIG. 35 depicts the effects of compound SL-11294 (CGC-11294) on cell motility.

FIG. 36 depicts the effects of compound SL-11184 (CGC-11184) on cell motility.

FIG. 37 depicts the effects of compound SL-11258 (CGC-11258) on cell motility.

FIG. 38 depicts the effects of compound SL-11177 (CGC-11177) on cell motility.

### DETAILED DESCRIPTION OF THE INVENTION

The term “a disease involving cell migration or cell motility” refers to a disease which can be treated by administering a compound which has a significant effect on cell migration or cell motility, or which modulates cell migration or cell motility. Motility or migration of any one or more types of cell in the subject or patient can be affected.

When the invention is used to modulate cell motility or cell migration, the cell motility or cell migration can occur or be modulated in vivo, in vitro, or ex vivo, where ex vivo refers to in vitro treatment of material of biological origin which is intended to be returned to an in vivo environment.

A “subject” or a “patient” refers to a vertebrate, preferably a mammal, more preferably a human. The polyamine analogs described herein or incorporated by reference herein are used to treat vertebrates, preferably mammals, more preferably humans.

“Treating” or “to treat” a disease using the methods of the invention is defined as administering one or more polyamine analogs, with or without additional therapeutic agents, in order to palliate, ameliorate, stabilize, reverse, slow, delay, prevent, reduce, or eliminate either the disease or the symptoms of the disease, or to retard or stop the progression of the disease or of symptoms of the disease. “Therapeutic use” of the polyamine analogs is defined as using one or more polyamine analogs to treat a disease, as defined above. A “therapeutically effective amount” is an amount sufficient to treat a disease, as defined above.

By “polyamine analog” is meant an organic cation structurally similar but non-identical to naturally occurring polyamines such as spermine and/or spermidine and their precursor, diamine putrescine. By a “polyamine”, a term well-understood in the art, is meant any of a group of aliphatic, straight-chain amines derived biosynthetically from amino acids; polyamines are reviewed in Marton et al. (1995) Ann. Rev. Pharm. Toxicol. 35:55-91. Polyamine analogs can be branched or un-branched. Polyamine analogs include, but are not limited to, BE-4444 [1,19-bis (ethylamino)-5,10,15-triazanonadecane]; BE-333 [N1,N11-diethylnorspermine; DENSPM; 1,11-bis(ethylamino)-4,8-diazaundecane; thermine; Warner-Parke-Davis]; BE-33 [N1,N7-bis(ethyl) norspermidine]; BE-34 [N1,N8-bis(ethyl)spermidine]; BE-44 [N1,N9-bis(ethyl) homospermidine]; BE-343 [N1,N12-bis(ethyl)spermine; diethylspermine-N-1-N12; DESPM]; BE-373 [N,N′-bis(3-ethylamino) propyl)-1,7-heptane diamine, Merrell-Dow]; BE-444 [N1,N14-bis(ethyl) homospermine; diethylhomospermine-N1-N14]; BE-3443 [1,17-bis(ethylamino)-4,9,14-triazaheptadecane]; and BE-4334 [1,17-bis(ethylamino)-5,9,13-triazaheptadecane]; 1,12-Me₂-SPM [1,12-dimethylspermine]. See also Feuerstein et al. (1991); Gosule et al. (1978) J. Mol. Biol. 121:311-326; Behe et al. (1981) Proc. Natl. Acad. Sci. USA 78:1619-23; Jain et al. (1989) Biochem. 28:2360-2364; Basu et al. (1990) Biochem. J 269:329-334; Porter et al. (1988), Advances in Enzyme Regulation, Pergamon Press, pp. 57-79; Frydman et al. (1992) Proc. Natl. Acad. Sci. USA 89:9186-9191; and Fernandez et al. (1994) Cell Mol. Biol. 40: 933-944.

By “conformationally restricted” is meant that, in a polyamine analog, at least two amino groups in the molecule are locked or limited in spatial configuration relative to each other. The amino groups within the molecule may be primary, secondary, tertiary, or quartenary, and are preferably primary or secondary amino groups, more preferably secondary amino groups. The relative movement of two amino groups can be restricted, for example, by incorporation of a cyclic or unsaturated moiety between them (exemplified, but not limited to, a ring, such as a three-carbon ring, four-carbon ring, five-carbon-ring, six-carbon ring, or a double or triple bond, such as a double or triple carbon bond). Groups restricting conformational flexibility by means of steric hindrance, yet favorable to the therapeutic effects of the compound, can also be used. A conformationally restricted polyamine analog can comprise at least two amino groups which are conformationally restricted relative to each other; a polyamine analog can also further comprise amino groups which are not conformationally restricted relative to other amino groups. Flexible molecules such as spermine and BE-444 can have a myriad of conformations and are therefore not conformationally restricted. Conformationally restricted polyamine analogs include, but are not limited to, the compounds disclosed in International Patent Application WO 98/17624, U.S. Pat. No. 5,889,061, and U.S. Pat. No. 6,392,098; the compounds disclosed in WO 00/66587 and U.S. Pat. No. 6,794,545; and the compounds disclosed in United States Patent Application Publication Nos. 2003/0072715, 2003/0195377, and International Patent Applications WO 02/10142, and WO 03/050072. Several of these compounds are depicted below in Table 1. All of the polyamine analog compounds (both conformationally restricted polyamine analog compounds and non-conformationally restricted polyamine analog compounds) disclosed in those patents or patent applications, and all other compounds comprising derivatives or conjugates of polyamine compounds disclosed in those patents or patent applications, including but not limited to those disclosed in the specification, claims, tables, examples, figures, and schemes of those patents or patent applications, are expressly incorporated by reference herein as compounds useful in the invention. The conformationally restricted polyamine analog compounds disclosed in those patents or patent applications, including but not limited to the specification, claims, tables, examples, figures, and schemes of those patents or patent applications, are expressly incorporated by reference herein as compounds useful in the invention.

In certain embodiments, the saturated oligoamines disclosed in U.S. Patent Application Publication No. 2003/0130356 can be used for treatment of diseases involving cell migration or cell motility, or to modulate or affect cell migration or cell motility, and all oligoamine compounds disclosed therein, including but not limited to those disclosed in the specification, claims, tables, examples, figures, and schemes of that patent application, are expressly incorporated by reference herein as compounds useful in the invention.

In certain additional embodiments, the polyamine analog-porphyrin conjugates disclosed in WO 00/66587 and U.S. Pat. No. 6,794,545, and WO 2004/02991 and U.S. Patent Application Publication No. 2004/0152687, can be used for treatment of diseases involving cell migration or cell motility, or to modulate or affect cell migration or cell motility, and all polyamine analog-porphyrin conjugates disclosed therein, including but not limited to those disclosed in the specification, claims, tables, examples, figures, and schemes of those patent applications, are expressly incorporated by reference herein as compounds useful in the invention.

In certain additional embodiments, the polyamines and the polyamine analog-peptide conjugates disclosed in U.S. Pat. No. 6,649,587 can be used for treatment of diseases involving cell migration or cell motility, or to modulate or affect cell migration or cell motility, and all polyamines and polyamine analog-peptide conjugates disclosed therein, including but not limited to those disclosed in the specification, claims, tables, examples, figures, and schemes of that patent, are expressly incorporated by reference herein as compounds useful in the invention.

In certain additional embodiments, the polyamine analog-amino acid conjugates disclosed in International Patent Application WO 02/38105 can be used for treatment of diseases involving cell migration or cell motility, or to modulate or affect cell migration or cell motility, and all polyamine analog-amino acid conjugates disclosed therein, including but not limited to those disclosed in the specification, claims, tables, examples, figures, and schemes of that patent application, are expressly incorporated by reference herein as compounds useful in the invention.

The invention includes the use of all of the compounds described herein or incorporated by reference herein, including any and all stereoisomers, salts, hydrates and solvates of the compounds described herein or incorporated by reference herein. The invention also includes the use of all compounds described herein or incorporated by reference herein in their non-salt, non-hydrate/non-solvate form. Particularly preferred are pharmaceutically acceptable salts. Pharmaceutically acceptable salts are those salts which retain the biological activity of the compound and which are not biologically or otherwise undesirable. The desired salt may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of the compounds with amino acids, such as aspartate salts and glutamate salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

The term “alkyl” refers to saturated aliphatic groups including straight-chain, branched-chain, cyclic groups, and combinations thereof, having the number of carbon atoms specified, or if no number is specified, having up to 12 carbon atoms, with preferred subsets of alkyl groups including C₁-C₁₂, C₁-C₁₀, C₁-C₈, C₁-C₆, and C₁-C₄ alkyl groups. “Straight-chain alkyl” or “linear alkyl” groups refers to alkyl groups that are neither cyclic nor branched, commonly designated as “n-alkyl” groups. Examples of alkyl groups include, but are not limited to, groups such as methyl, ethyl, n-propyl, isopropyl, butyl, n-butyl, isobutyl, sec-butyl, t-butyl, pentyl, n-pentyl, hexyl, heptyl, octyl, nonyl, decyl, undecyl, dodecyl, neopentyl, cyclopropyl, cyclobutyl, cyclopentyl, cyclohexyl, and adamantyl. Cyclic groups can consist of one ring, including, but not limited to, groups such as cycloheptyl, or multiple fused rings, including, but not limited to, groups such as adamantyl or norbornyl.

“Substituted alkyl” refers to alkyl groups substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups.

“Hydroxyalkyl” specifically refers to alkyl groups having the number of carbon atoms specified substituted with one —OH group. Thus, “C₃ linear hydroxyalkyl” refers to —CH₂CH₂CHOH—, —CH₂CHOHCH₂—, and —CHOHCH₂CH₂—.

The term “alkenyl” refers to unsaturated aliphatic groups including straight-chain (linear), branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃; and —CH₂—CH₂-cyclohexenyl, where the ethyl group can be attached to the cyclohexenyl moiety at any available carbon valence. The term “alkynyl” refers to unsaturated aliphatic groups including straight-chain (linear), branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or multiple condensed rings (including, but not limited to, groups such as naphthyl or anthryl), and includes both unsubstituted and substituted aryl groups. “Substituted aryls” refers to aryls substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldelhyde, carboalkoxy and carboxamide, or a functionality that can be suitably blocked, if necessary for purposes of the invention, with a protecting group.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, 2-ethylthiophenyl, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, thiophene, or furyl) or multiple condensed rings (including, but not limited to, examples such as imidazolyl, indolizinyl or benzothienyl) and having at least one hetero atom, including, but not limited to, heteroatoms such as N, O, P, or S, within the ring. Unless otherwise specified, heteroalkyl, heteroalkenyl, heteroalkynyl, and heteroaryl groups have between one and five heteroatoms and between one and twelve carbon atoms. “Substituted heteroalkyl,” “substituted heteroalkenyl,” “substituted heteroalkynyl,” and “substituted heteroaryl” groups refer to heteroalkyl, heteroalkenyl, heteroalkynyl, and heteroaryl groups substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. If chemically possible, the heteroatom(s) as well as the carbon atoms of the group can be substituted. The heteroatom(s) can also be in oxidized form, if chemically possible.

The term “alkylaryl” refers to an alkyl group having the number of carbon atoms designated, appended to one, two, or three aryl groups.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, and t-butoxy.

The term “alkanoate” as used herein refers to an ionized carboxylic acid group, such as acetate(CH₃C(═O)—O⁽⁻¹⁾), propionate (CH₃CH₂C(═O)—O⁽⁻¹⁾), and the like. “Alkyl alkanoate” refers to a carboxylic acid esterified with an alkoxy group, such as ethyl acetate(CH₃C(═O)—O—CH₂CH₃). “ω-haloalkyl alkanoate” refers to an alkyl alkanoate bearing a halogen atom on the alkanoate carbon atom furthest from the carboxyl group; thus, ethyl ω-bromo propionate refers to ethyl 3-bromopropionate, methyl ω-chloro n-butanoate refers to methyl 4-chloro n-butanoate, etc.

The terms “halo” and “halogen” as used herein refer to Cl, Br, F or I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York,). Amino protecting groups include, but are not limited to, mesitylenesulfonyl (Mes), benzyloxycarbonyl (CBz or Z), t-butyloxycarbonyl (Boc), t-butyldimethylsilyl (TBDIMS or TBDMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Hydroxyl protecting groups include, but are not limited to, Fmoc, TBDIMS, photolabile protecting groups (such as nitroveratryl oxymethyl ether (Nvom)), Mom (methoxy methyl ether), and Mem (methoxy ethoxy methyl ether), NPEOC (4-nitrophenethyloxycarbonyl) and NPEOM (4-nitrophenethyloxymethyloxycarbonyl).

Examples of compounds useful in the invention are depicted in Table 1. While some of the compounds are depicted as salts, such as the hydrochloride salt, it is to be understood that the disclosure in the table embraces all salts, hydrates, and solvates of the compounds depicted therein, as well as the non-salt, non-hydrate/non-solvate form of the compound, as is well understood by the skilled artisan. Table 1 includes both non-conformationally restricted polyamine analogs and conformationally restricted polyamine analogs. While both types of polyamine analogs are useful in the invention, the conformationally restricted polyamine analogs are preferred for use in the invention.

TABLE 1

| Compound | Structure |
|---|---|
| SL-11027 | 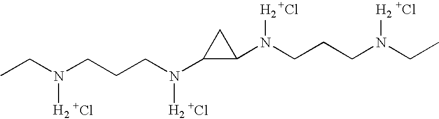 |
| SL-11028 | 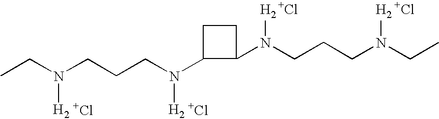 |
| SL-11029 | 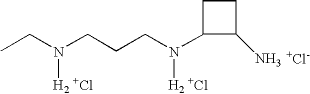 |
| SL-11033 | 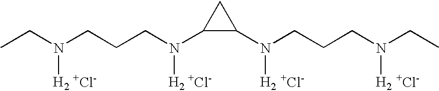 |
| SL-11034 | 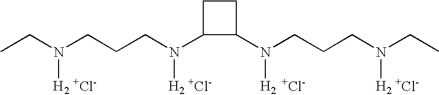 |
| SL-11035 | 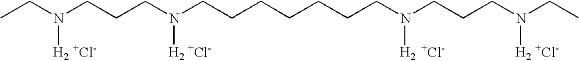 |
| SL-11036 | 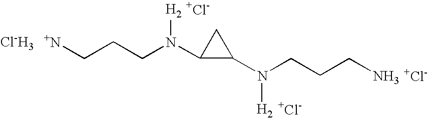 |
| SL-11037 | 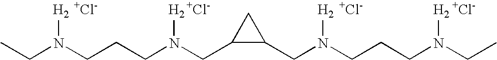 |
| SL-11038 | 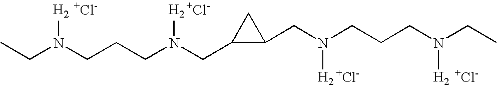 |
| SL-11043 | 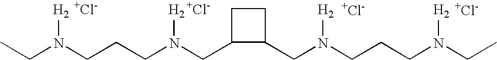 |
| SL-11044 | 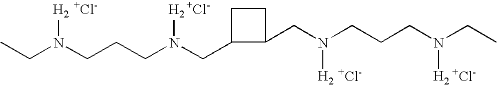 |
| SL-11047 | 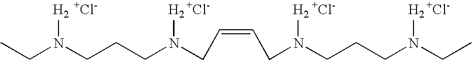 |
| SL-11048 | 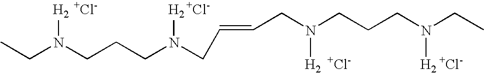 |
| SL-11050 | BnNH(CH₂)₄NHBn |
| | •2HCl |
| SL-11061 | EtNH(CH₂)₄NH(CH₂)₄NH(CH₂)₄NH(CH₂)₄—NHEt•5HCl |
| SL-11093 | 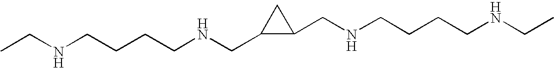 |
| SL-11094 | 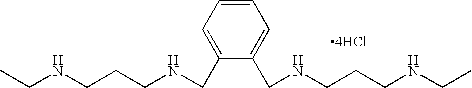 |
| SL-11098 | 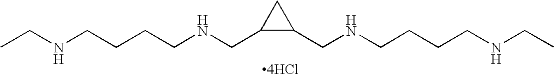 |
| SL-11099 | 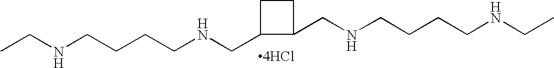 |
| SL-11100 | 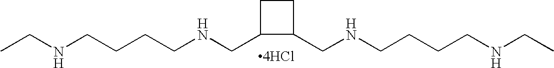 |
| SL-11101 | 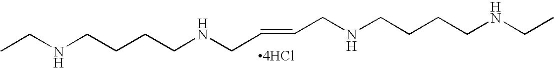 |
| SL-11102 | 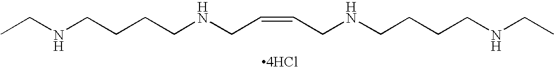 |
| SL-11103 | 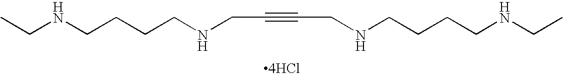 |
| SL-11104 | 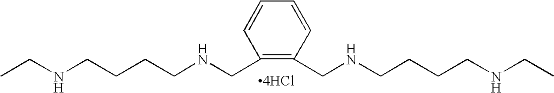 |
| SL-11105 | 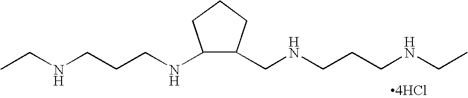 |
| SL-11108 | 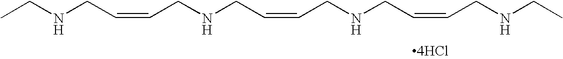 |
| SL-11114 | 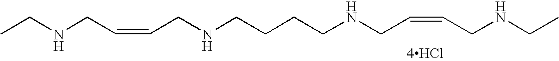 |
| SL-11119 | 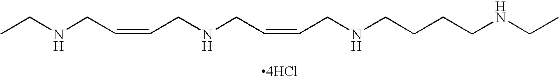 |
| SL-11090 | 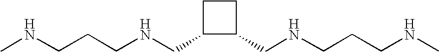 |
| SL-11091 | 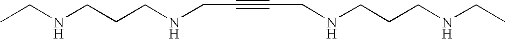 |
| SL-11092 | 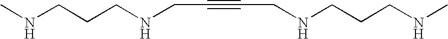 |
| SL-11101 | 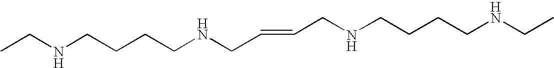 |
| SL-11103 | 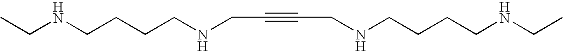 |
| SL-11114 | 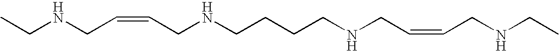 |
| SL-11118 | 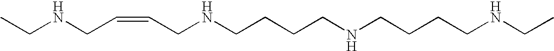 |
| SL-11121 | 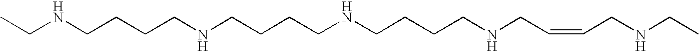 |
| SL-11122 | 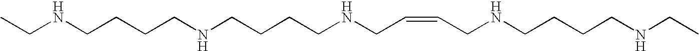 |
| SL-11123 | 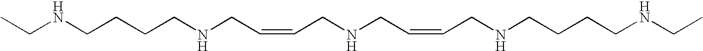 |
| SL-11124 | 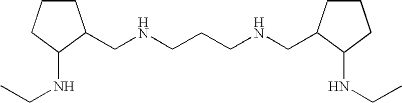 |
| SL-11126 | 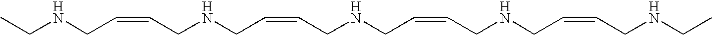 |
| SL-11127 | 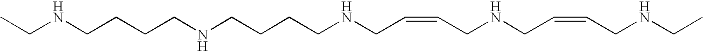 |
| SL-11128 | 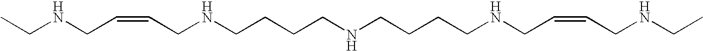 |
| SL-11129 | 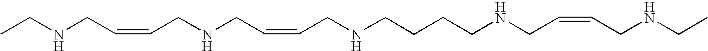 |
| SL-11130 | 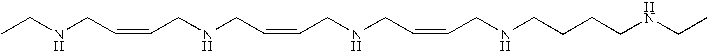 |
| SL-11132 | 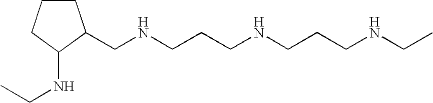 |
| SL-11133 | 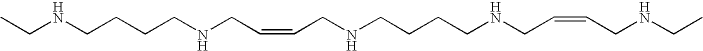 |
| SL-11134 | 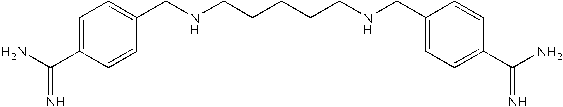 |
| SL-11135 | 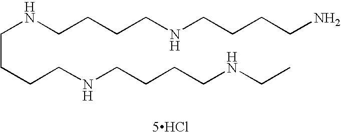 |
| SL-11136 | 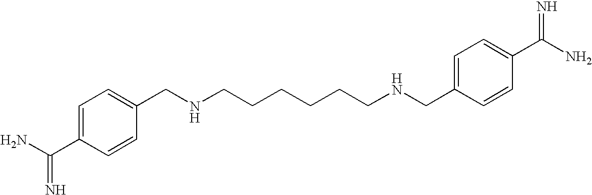 |
| SL-11137 | 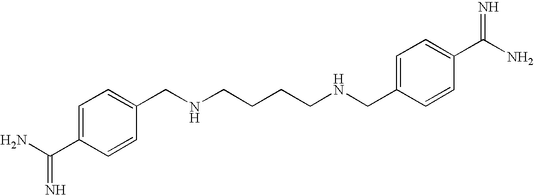 |
| SL-11141 | 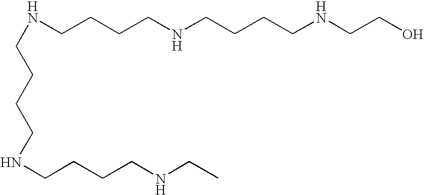 |
| SL-11143 | 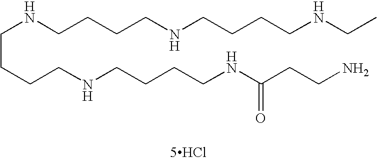 |
| SL-11144 | 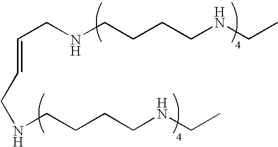 |
| SL-11150 | 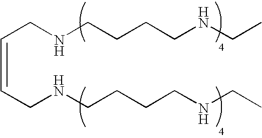 |
| SL-11155 | 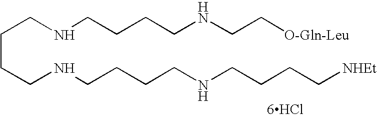 |
| SL-11157 | 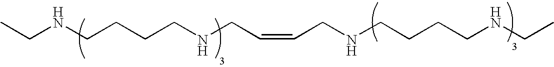 |
| SL-11158 | 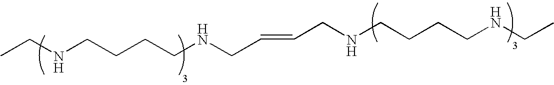 |
| SL-11201 | 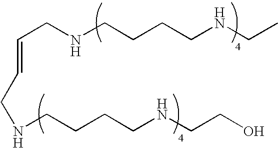 |
| SL-11202 | 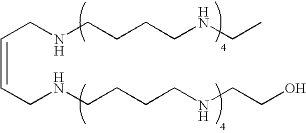 |
| SL-11174 | 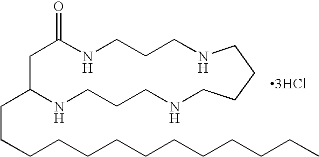 |
| SL-11197 | 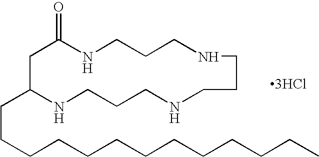 |
| SL-11199 | 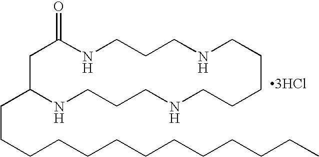 |
| SL-11200 | 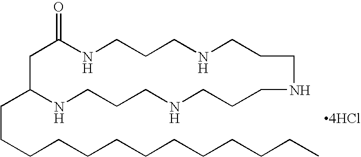 |
| SL-11208 | 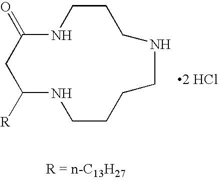 |
| SL-11238 | 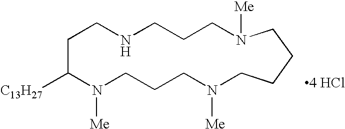 |
| SL-11239 | 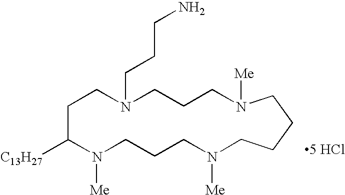 |

Syntheses of various compounds useful in the invention are found in the following United States patents: U.S. Pat. No. 5,889,061, U.S. Pat. No. 6,392,098, U.S. Pat. No. 6,649,587, and U.S. Pat. No. 6,794,545; in the following United States Patent Application Publications: U.S. 2003/0072715, U.S. 2003/0130356, U.S. 2003/0195377, U.S. 2004/0152687; and in International Patent Applications WO 98/17624, WO 00/66587, WO 02/10142, WO 02/38105, WO 03/050072, and WO 2004/02991. For example, methods for synthesis of SL-11144 and SL-11150 (CGC-11144 and CGC-11150) are found in U.S. Pat. No. 6,794,545; methods for synthesis of SL-11175 and SL-11226 (CGC-11175 and CGC-11226) are found in U.S. 2003/0130356; methods for synthesis of SL-11099 and SL-11102 (CGC-11099 and CGC-11102) are found in U.S. Pat. No. 5,889,061 and U.S. Pat. No. 6,649,587; methods for synthesis of SL-11172 (CGC-11172) are found in U.S. Pat. No. 6,794,545; methods for synthesis of SL-11288, SL-11184 and SL-11177 (CGC-11288, CGC-11184 and CGC-11177) are found in U.S. Pat. No. 6,794,545; and a method of synthesis of SL-11255 (CGC-11255) is given below in Example 1.

### Diseases Involving Cell Motility or Cell Migration

The invention embraces methods of treating a variety of diseases involving cell migration or cell motility, such as inflammation, infection (i.e., invasion of the body by bacteria, fungi, viruses, or other microorganisms), endometriosis, abnormal immune responses, undesired angiogenesis, tumor cell metastasis or invasion, atherosclerosis, vascular graft occlusion, restenosis (e.g., subsequent to angioplasty), transplant rejection, other complications of transplants, and glomerilonephritis (which can be caused by undesired mesangial cell migration).

Inflammatory diseases involving cell migration or cell motility which can be treated include, but are not limited to, arthritis; inflammatory responses subsequent to stroke, ischemia, or reperfusion; inflammatory bowel diseases; gingivitis and periodontitis; and asthma. Additional diseases involving cell migration or cell motility which can be treated are diabetes, inflammatory aortic aneurysm, cancer, rheumatic fever, systemic lupus erythematosus, Reiter's syndrome, ankylosing spondylitis, ulcerative colitis, Crohn's disease, pelvic inflammatory disease, multiple sclerosis, osteomyelitis, adhesive capsulitis, rheumatoid arthritis, psoriatic arthritis, oligoarthritis, osteoarthritis, periarthritis, polyarthritis, coxarthritis, psoriasis, Still's disease, synovitis, Alzheimer's disease, Parkinson's disease, amyotrophic lateral sclerosis, osteoporosis, inflammatory dermatosis and wound healing. In additional embodiments, diseases involving cell migration or cell motility which can be treated include asbestosis or silicosis, liver cirrhosis, pemphigus vulgaris, polymyositis-dermatomyositis, Sjogren's syndrome, Lyme disease, Behcet's disease. Acute inflammatory diseases involving cell migration or cell motility, such as bursitis, synovitis, capsulitis, tendinitis and other inflammatory conditions due to trauma can also be treated.

### Modes of Administration

Compounds useful in the methods of the invention can be administered to a patient or subject (preferably a human patient or subject) via any route known in the art, including, but not limited to, those disclosed herein. Methods of administration include, but are not limited to, systemic, transpleural, intravenous, oral, intraarterial, intramuscular, topical, via inhalation (e.g. as mists or sprays), via nasal mucosa, subcutaneous, transdermal, intraperitoneal, gastrointestinal, rectal, and via administration to various tissues or organs such as the eye or ear. The compounds described or incorporated by reference for use herein can be administered in the form of tablets, pills, powder mixtures, capsules, granules, injectables, creams, solutions, suppositories, enemas, emulsions, dispersions, mouthwashes, food premixes, and in other suitable forms. The compounds can also be administered in liposome formulations. The compounds can also be administered as prodrugs, where the prodrug undergoes transformation in the treated subject to a form which is therapeutically effective. The compounds can also be administered to the subject or patient as an implant. Preferred implants are biocompatible and/or biodegradable sustained release formulations which gradually release the compounds over a period of time. Additional methods of administration are known in the art.

The pharmaceutical dosage form which contains the compounds for use in the invention is conveniently admixed with a non-toxic pharmaceutical organic carrier or a non-toxic pharmaceutical inorganic carrier. Typical pharmaceutically-acceptable carriers include, for example, mannitol, ethanol and other alcohols, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the compound(s) to retain its pharmacological activity in the body. Formulations for parenteral and nonparenteral drug delivery are known in the art and are set forth in Remington: The Science and Practice of Pharmacy, 20th Edition, Lippincott, Williams & Wilkins (2000). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; desiccants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tableting lubricants, for example magnesium stearate, talc, polyethylene glycol or silica; disintegrants, for example potato starch; or acceptable wetting agents such as sodium lauryl sulfate. The tablets can be coated according to methods well known in standard pharmaceutical practice. Liquid forms for ingestion can be formulated using known liquid carriers, including aqueous and non-aqueous carriers such as sterile water, sterile saline, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, the compounds for use in the invention can be administered as injectable dosages of a solution or suspension of the compound in a physiologically acceptable diluent or sterile liquid carrier such as water, saline, or oil, with or without additional surfactants or adjuvants. An illustrative list of carrier oils would include animal and vegetable oils (e.g., peanut oil, soy bean oil), petroleum-derived oils (e.g., mineral oil), and synthetic oils. For injectable unit doses, sterile liquids such as water, saline, aqueous dextrose and related sugar solutions are preferred liquid carriers.

The pharmaceutical unit dosage chosen is preferably fabricated and administered to provide a defined final concentration of drug either in the blood, or in tissues where the disease involving cell motility or cell migration is localized. The optimal effective concentration of the compounds of the invention can be determined empirically and will depend on the type and severity of the disease, route of administration, disease progression and health, mass and body area of the patient. Such determinations are within the skill of one in the art. Examples of dosages which can be used include, but are not limited to, an effective amount within the dosage range of about 0.1 μg/kg to about 300 mg/kg, or within about 1.0 μg/kg to about 40 mg/kg body weight, or within about 10 μg/kg to about 20 mg/kg body weight, or within about 0.1 mg/kg to about 20 mg/kg body weight, or within about 1 mg/kg to about 20 mg/kg body weight, preferably between about 0.1 μg/kg to about 10 mg/kg body weight. The dosages may be administered in a single daily dose, or the total daily dosage may be administered in divided dosage of two, three or four times daily. Dosages may also be administered less frequently than daily, for example, six times a week, five times a week, four times a week, three times a week, twice a week, or once a week. The dosages may also be administered in a sustained release formulation, such as in an implant which gradually releases the compounds for use in the invention over a period of time, and which allow for the drug to be administered less frequently, such as once a month, once every 2-6 months, once every year, or even a single administration which need not be repeated. The sustained release devices (such as pellets, microspheres, and the like) may be administered by injection or surgically implanted in various locations in the body.

The compounds for use in the invention can be administered as the sole active ingredient, or can be administered in combination with another active ingredient.

### Kits

The invention also provides articles of manufacture and kits containing materials useful for treating diseases involving cell motility or cell migration. The article of manufacture comprises a container with a label. Suitable containers include, for example, bottles, vials, and test tubes. The containers may be formed from a variety of materials such as glass or plastic. The container holds a composition having an active agent which is effective for treating the disease involving cell motility or cell migration. The active agent in the composition is one or more polyamine analogs, such as one or more of the conformationally restricted polyamine analogs disclosed herein or incorporated by reference herein. The label on the container indicates that the composition is used for treating one or more diseases involving cell motility or cell migration, and may also indicate directions for use.

The invention also provides kits comprising any one or more of a polyamine analog. In some embodiments, the kit of the invention comprises the container described above. In other embodiments, the kit of the invention comprises the container described above and a second container comprising a buffer. It may further include other materials desirable from a commercial and user standpoint, including other buffers, diluents, filters, needles, syringes, and package inserts with instructions for performing any methods described herein (that is, methods for treating diseases involving cell motility or cell migration).

In other aspects, the kits may be used for any of the methods described herein, including, for example, to treat a patient or subject suffering from a disease involving cell motility or cell migration.

### EXAMPLES

### Example 1

### Synthesis of 1,2-bis((1-phenylcyclopropylaminobutyl)aminomethyl)cyclopropane (CGC-11255; SL-11255)

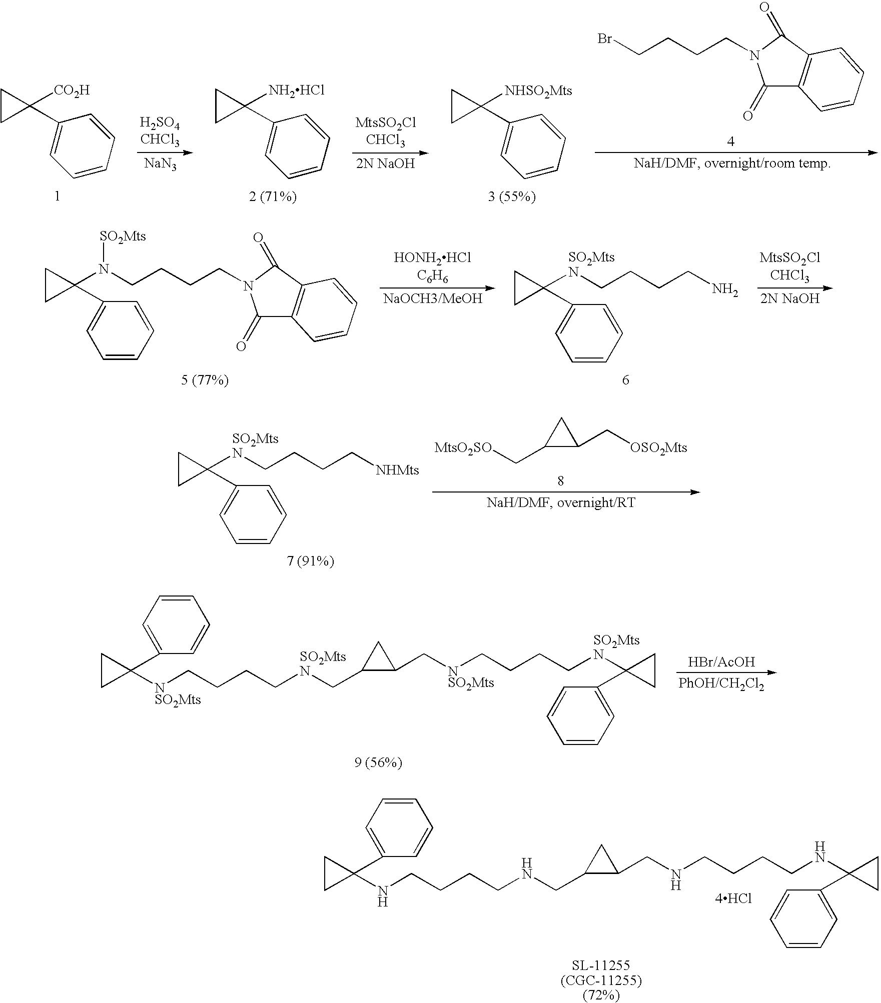

1-Phenylcyclopropane carboxylic acid (1) is reacted with sodium azide and sulfuric acid in CHCl₃ to yield 1-phenylcyclopropanamine (2). 1-Phenylcyclopropanamine is then reacted with 2-mesitylenesulfonyl chloride in chloroform under basic conditions to yield the protected derivative (3), N-(1-phenylcyclopropyl)mesitylenesulfonamide. N-(1-phenylcyclopropyl)mesitylenesulfonamide (3) is then reacted with N-(4-bromobutyl)phthalimide (4) in DMF with sodium hydride at room temperature overnight, to yield N-(2-mesitylenesulfonyl)-N-(1-phenylcyclopropyl)-4-phthalimidobutylamine (5).

The phthaloyl group is removed from N-(2-mesitylenesulfonyl)-N-(1-phenylcyclopropyl)-4-phthalimidobutylamine (5) by reaction with hydroxylamine hydrochloride in benzene and sodium methoxide in methanol, to produce N-(4-aminobutyl)-N-(1-phenylcyclopropyl)-2-mesitylenesulfonamide (6). The free amino group is then re-protected with 2-mesitylenesulfonyl chloride in 2N NaOH in CHCl₃ to give (7), N-(4-(mesitylene-2-sulfonylamino)butyl)-N-(1-phenylcyclopropyl)-2-mesitylenesulfonamide. (7) is then reacted with 1,2-bis(mesitylene-2-sulfonyloxymethyl)cyclopropane (8) in DMF with sodium hydride at room temperature overnight to yield (9), the tetra-mesitylene-2-sulfonyl-protected derivative of 1,2-bis((N-(1-phenylcyclopropyl)-4-aminobutyl)aminomethyl)cyclopropane. The mesitylene protecting groups are then removed using HBr in acetic acid and phenol in methylene chloride to yield 1,2-bis((N-(1-phenylcyclopropyl)-4-aminobutyl)aminomethyl)cyclopropane (CGC-11255; SL-11255).

Using the synthesis above, CGC-11255 was prepared and its effects on cell migration and cell motility were evaluated.

### Example 2

### Cell Migration Assay

Migration assays were performed using a proprietary 96-well monolayer migration assay. The 96-well platform is an adaptation of the monolayer radial migration assay (Berens, M E and Beaudry C., “Radial monolayer cell migration assay,” Methods Mol. Med. 88:219-24 (2004)). The improved platform allows high throughput screening of pharmacological agents for their effects on cell migration.

The 96 well slides were first coated with 0.1 μg/ml laminin, a motility stimulating substrate, in PBS for 1 hour at 37° C., rinsed three times in PBS, and then treated with 0.1% BSA in PBS for 1 hour at room temperature. Human glioblastoma SNB19 cells were plated at 3,000 cells per sedimentation channel and incubated overnight to allow cell sedimentation and attachment to the substrate. After removal of the manifold, the cells were treated with the compounds either at 0, 0.1 μM, 1 μM or 10M. The experiment was done in duplicate with appropriate controls on each slide. After adding the compounds, the diameter of each cell population was initially measured using an inverted microscope and image analysis software (Scion Image Corp, Frederick, Md.). Cells were incubated an additional 72 hours in the presence (or not) of the pharmacological compounds. The diameter of each cell population was again assessed at 24 hours, 48 hours and 72 hours to calculate the overall migration rate. Migration results were reported as the specific radial movement (μm/day) of the cell population. After each timepoint, if a compound was found to be active, images of the cell populations were captured. At the end of the assay, cells were rinsed with PBS, then treated with LIVE/DEAD reagent staining (Molecular Probes, Eugene, Oreg.) to assess compound toxicity.

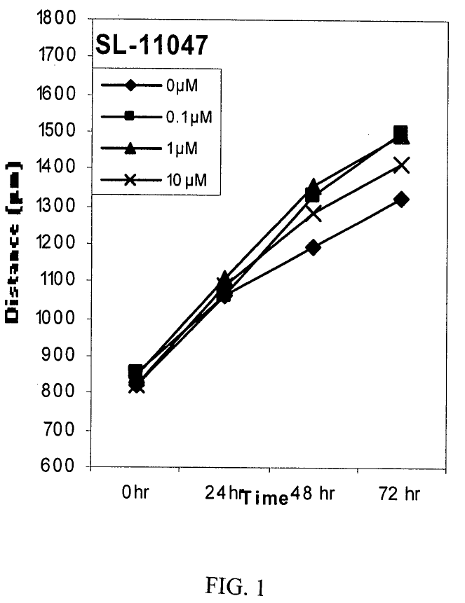

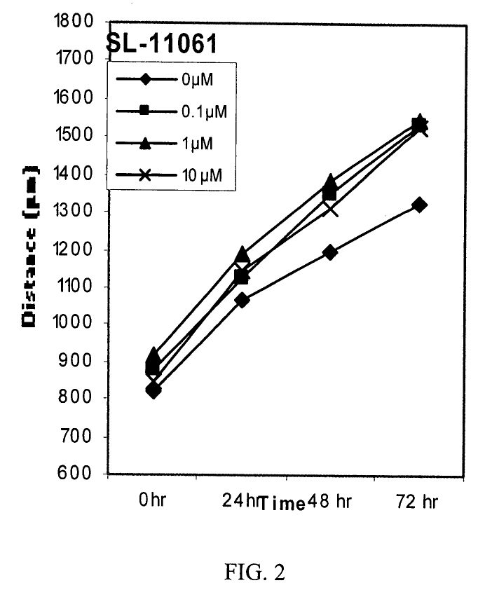

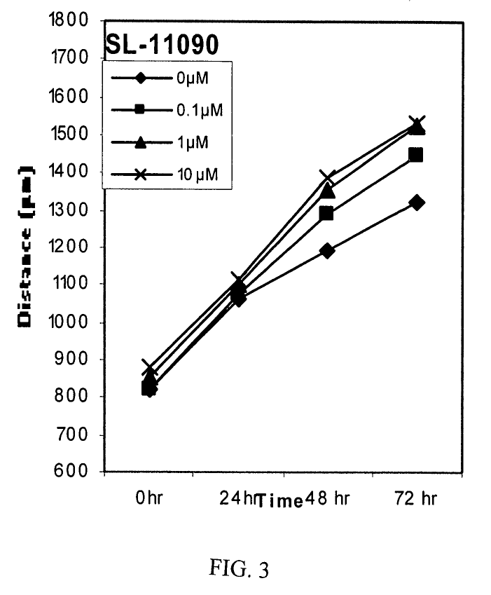

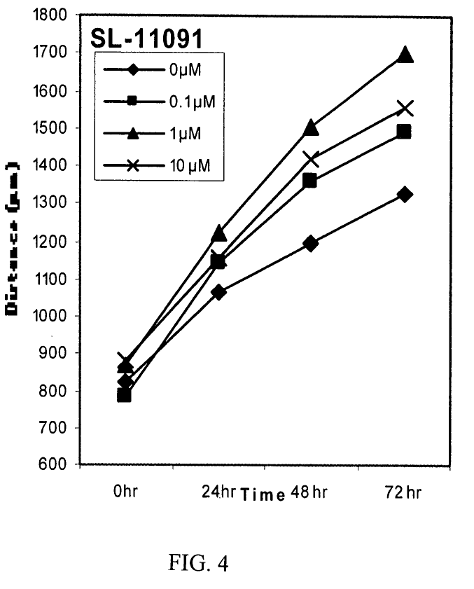

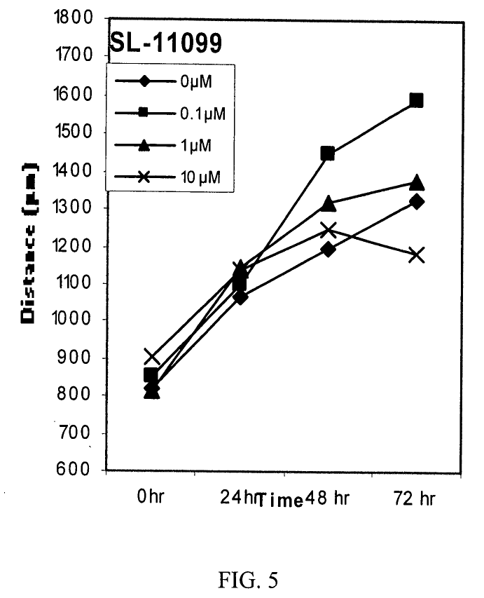

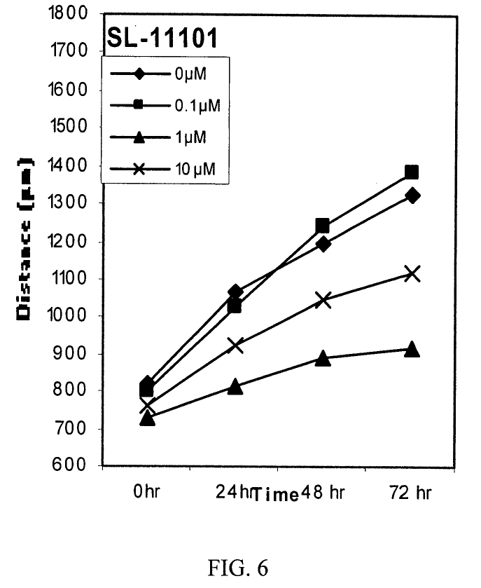

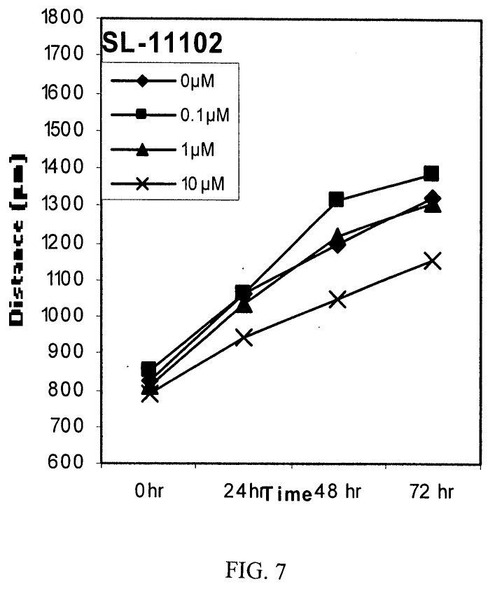

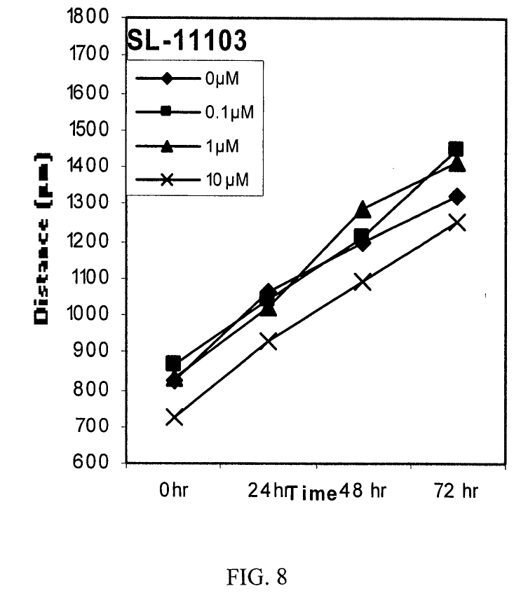

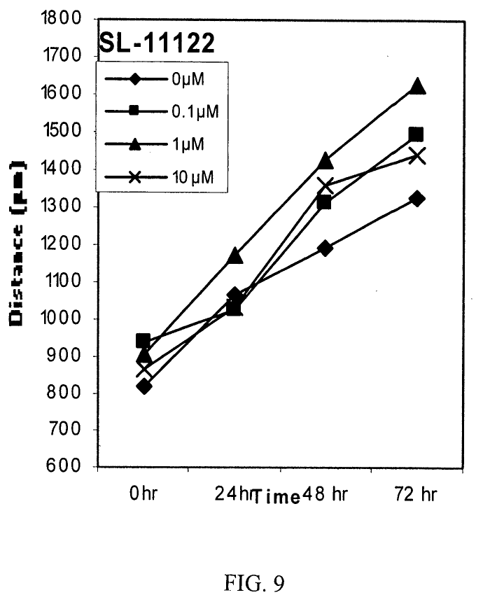

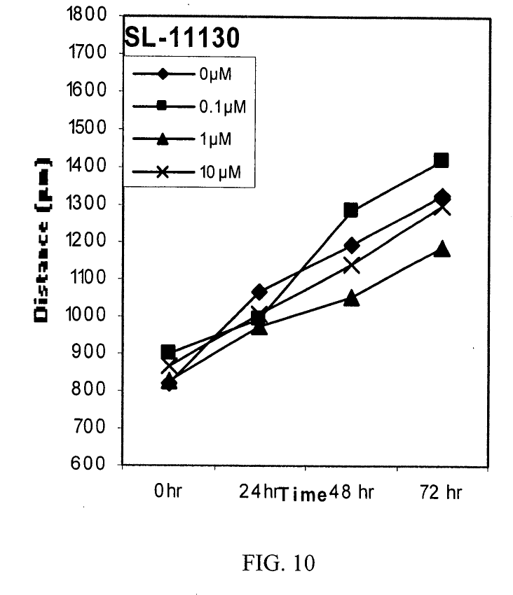

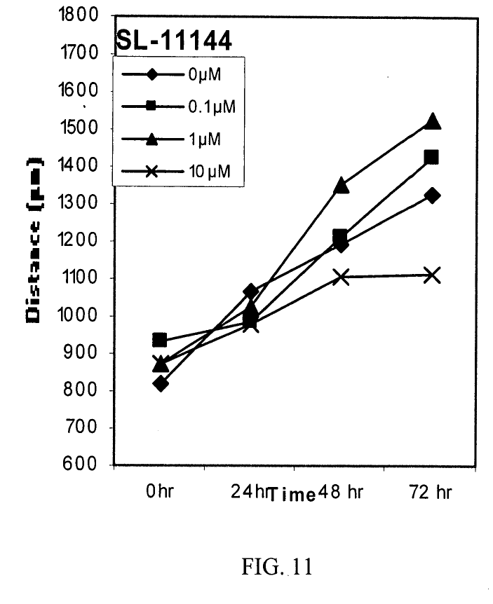

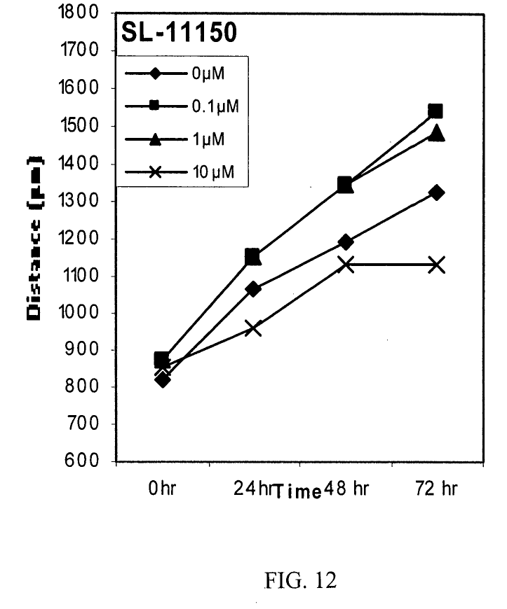

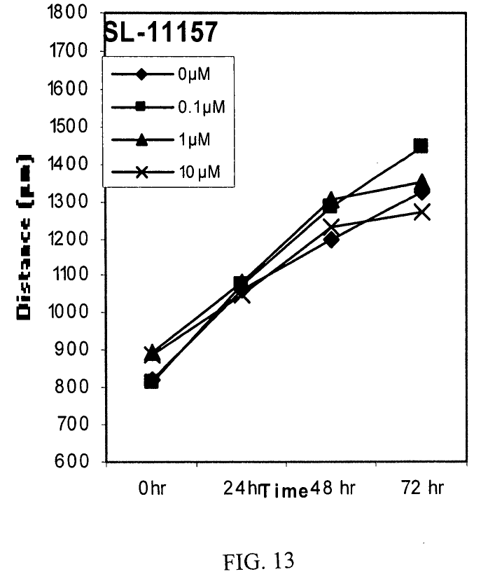

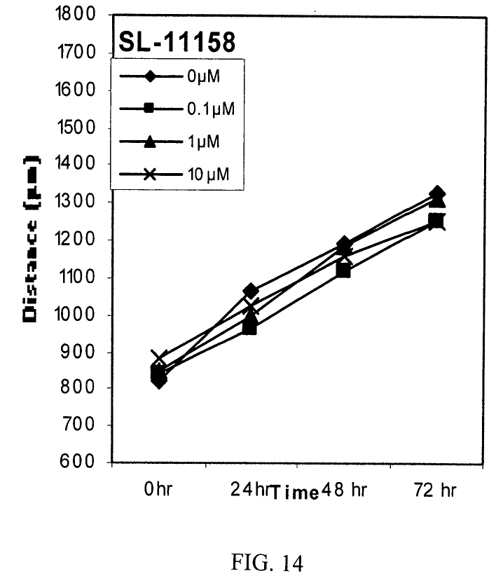

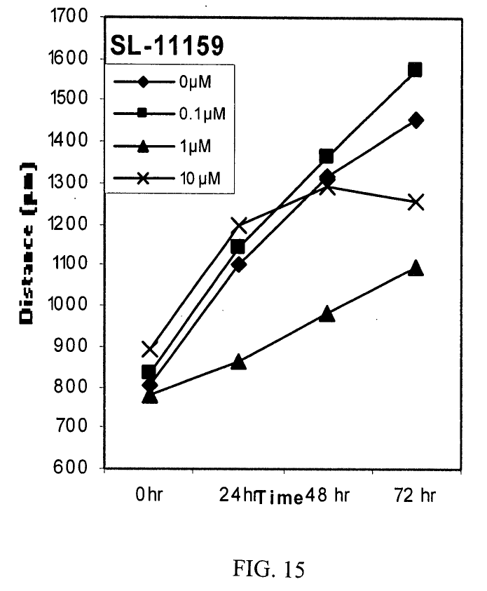

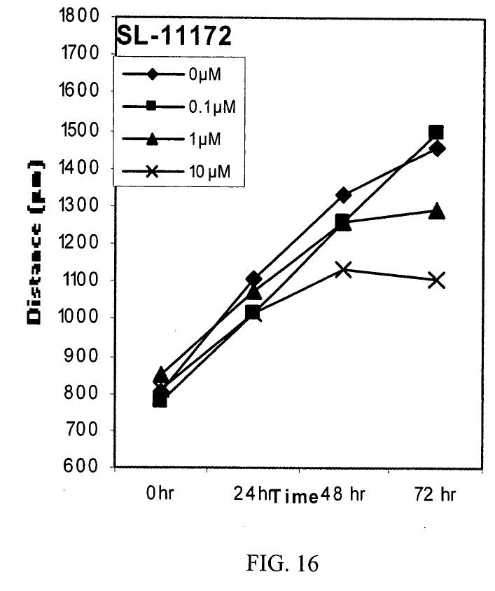

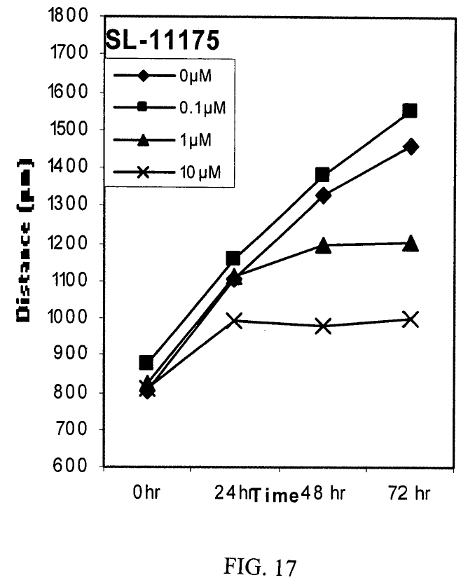

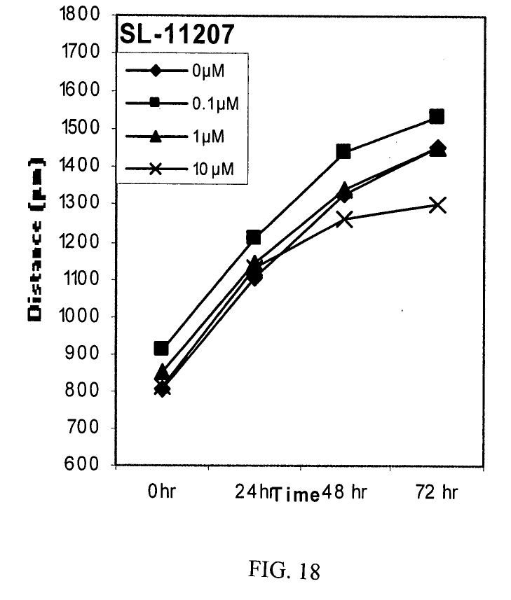

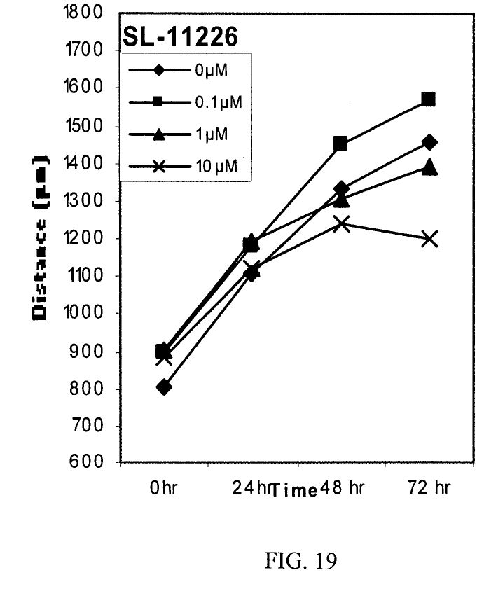

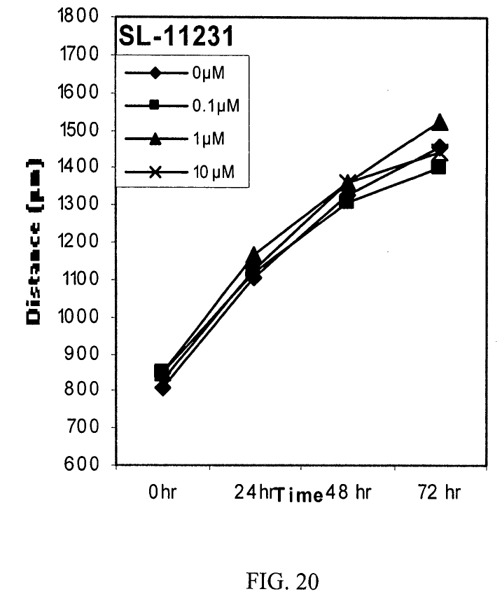

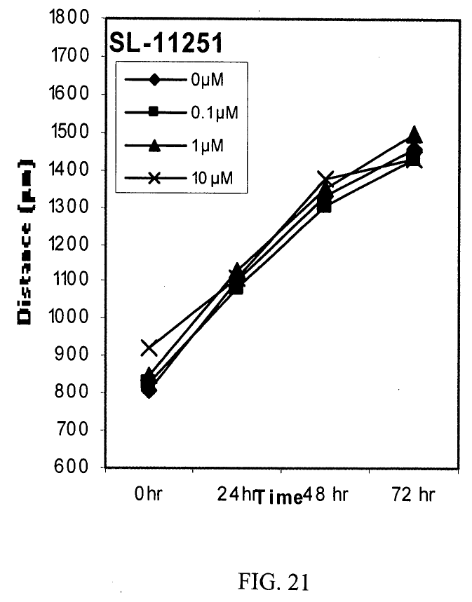

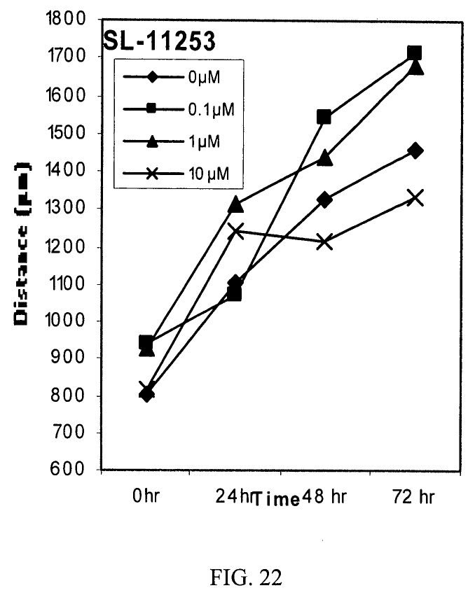

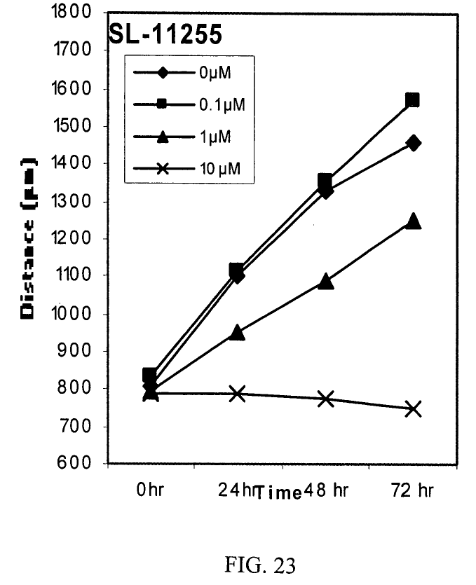

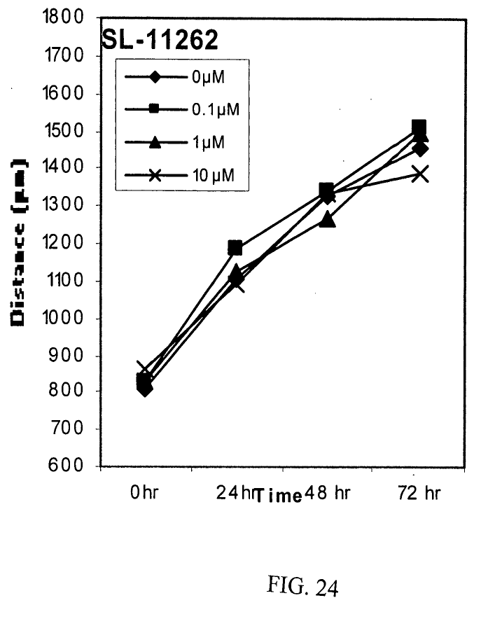

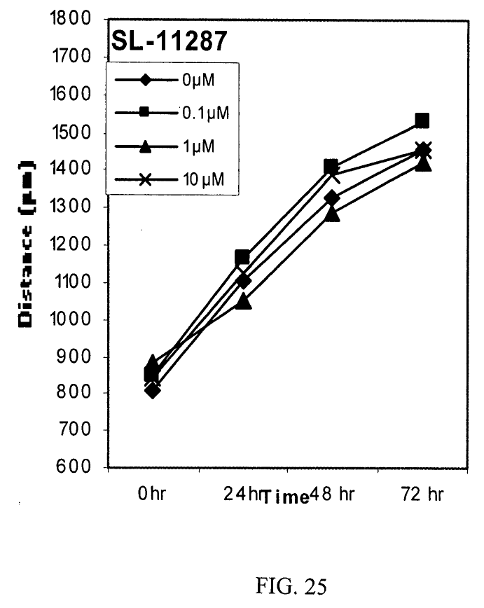

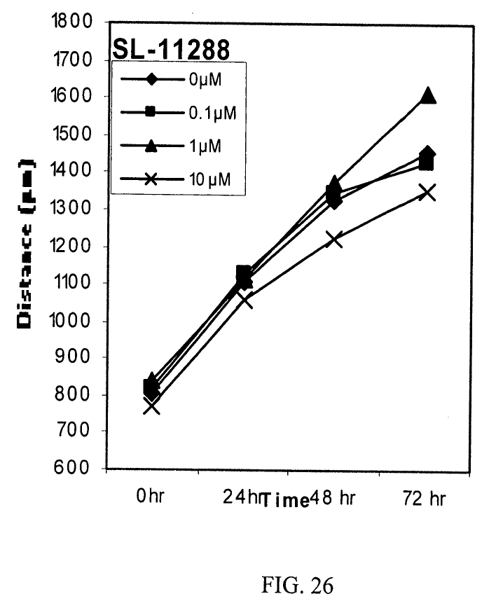

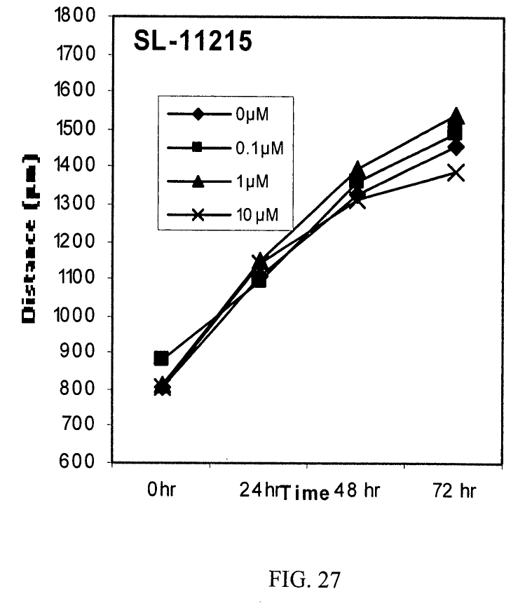

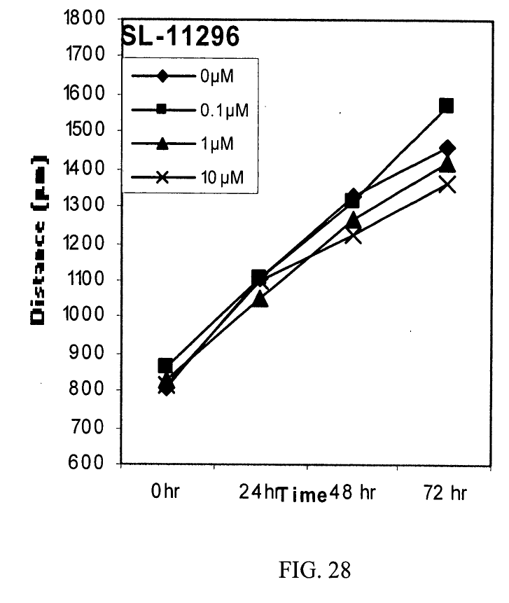

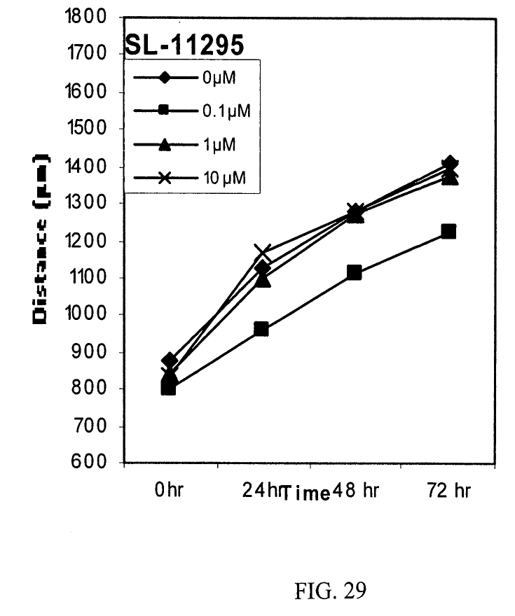

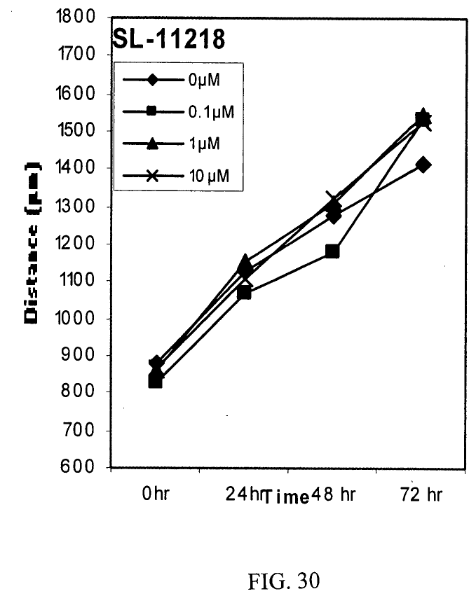

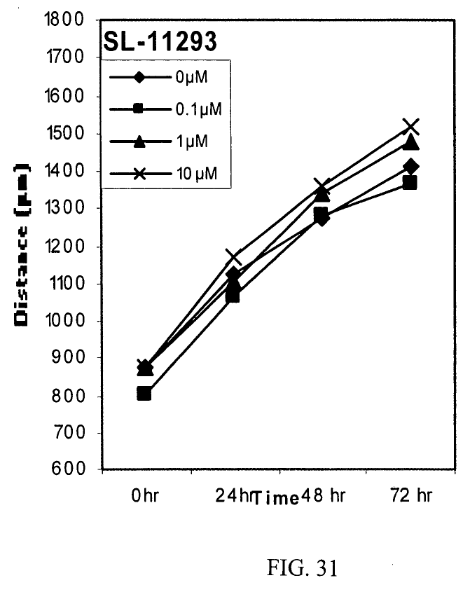

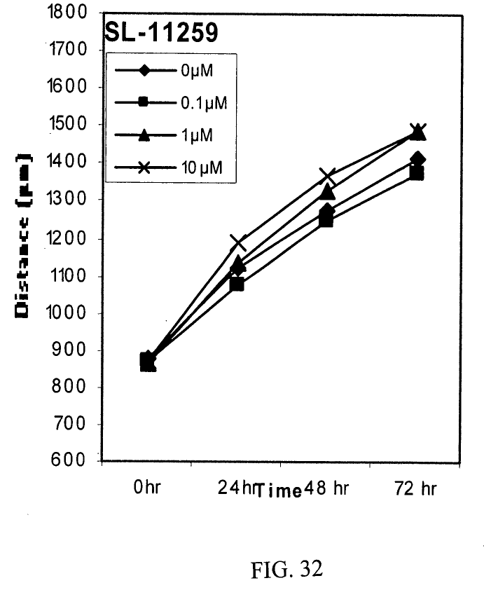

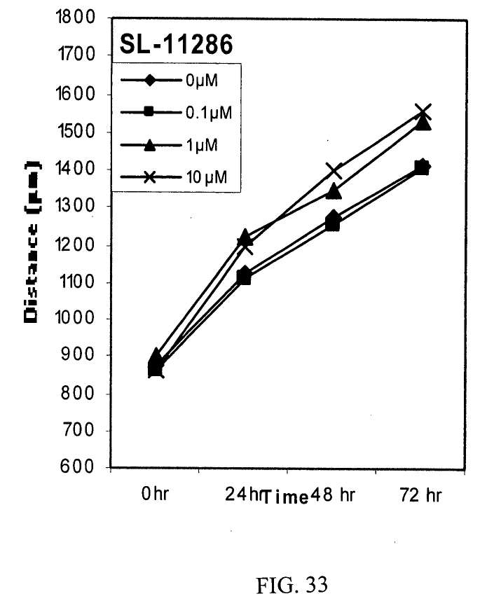

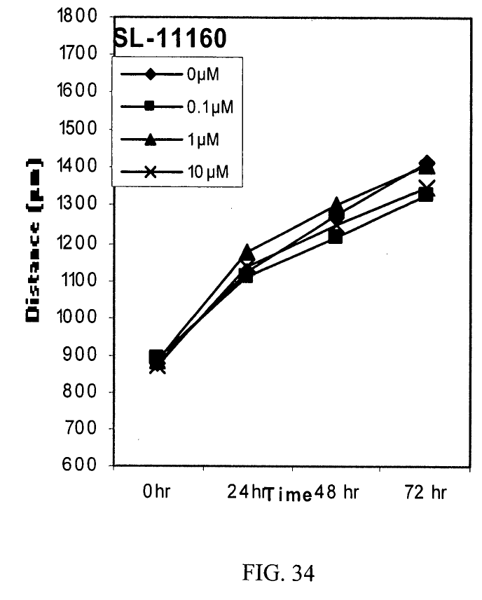

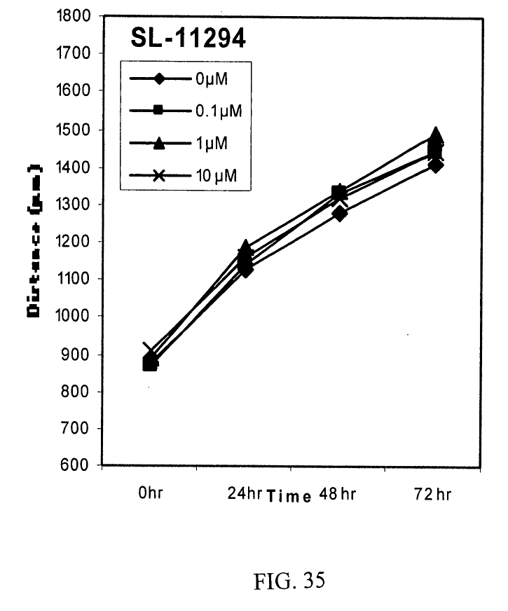

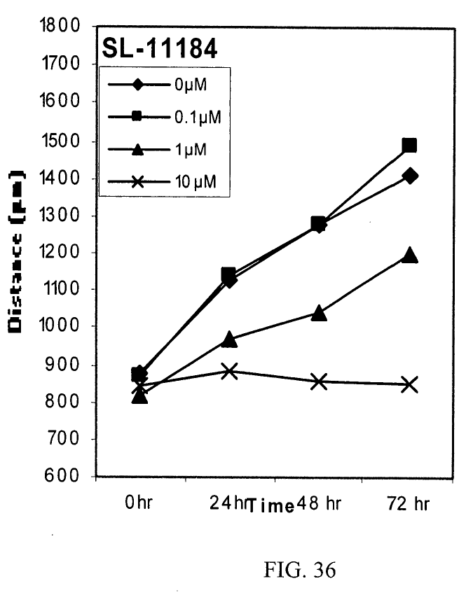

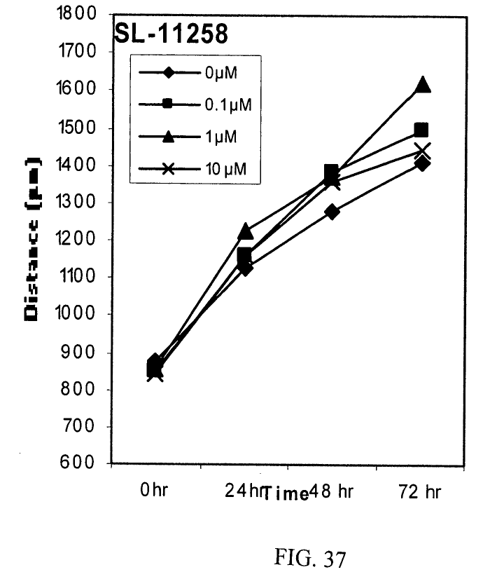

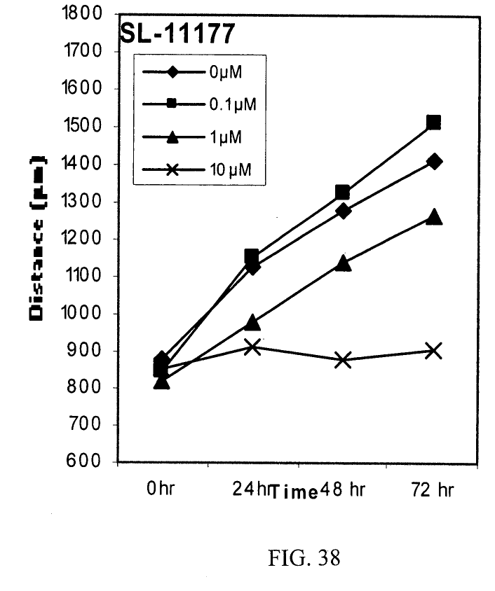

FIGS. 1-38 show data for the activity of compounds in the migration assay. Eleven compounds demonstrated activity in both replicates against the human glioblastoma SNB19 cell line. The data shown in the Figures represent the distance (μm) traveled by the cells over time in the presence of the compounds at different concentrations. Each Figure is derived from one representative replicate of the experiment rather then the average of both replicates. Time of onset of compound activity was observed as early as 2 hours after drug addition with some compounds; other agents did not manifest activity until 72 hours post drug addition. The Live/Dead assay revealed that 6 compounds had severe cytotoxic effects at 10 μM at time of staining (SL-11172, SL-11175, SL-11226, SL-11255, SL11184 and SL-11177). Those same compounds were not toxic at lower concentrations. None of the other active compounds showed significant signs of cytotoxicity compared to controls.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of inhibiting cell motility or cell migration, comprising: administering one or more polyamine analogs to a subject in an amount sufficient to inhibit cell motility or cell migration.

2. The method of claim 1, wherein the polyamine analog is a conformationally restricted polyamine analog.

3. The method of claim 1, wherein the polyamine analog is selected from the group consisting of: 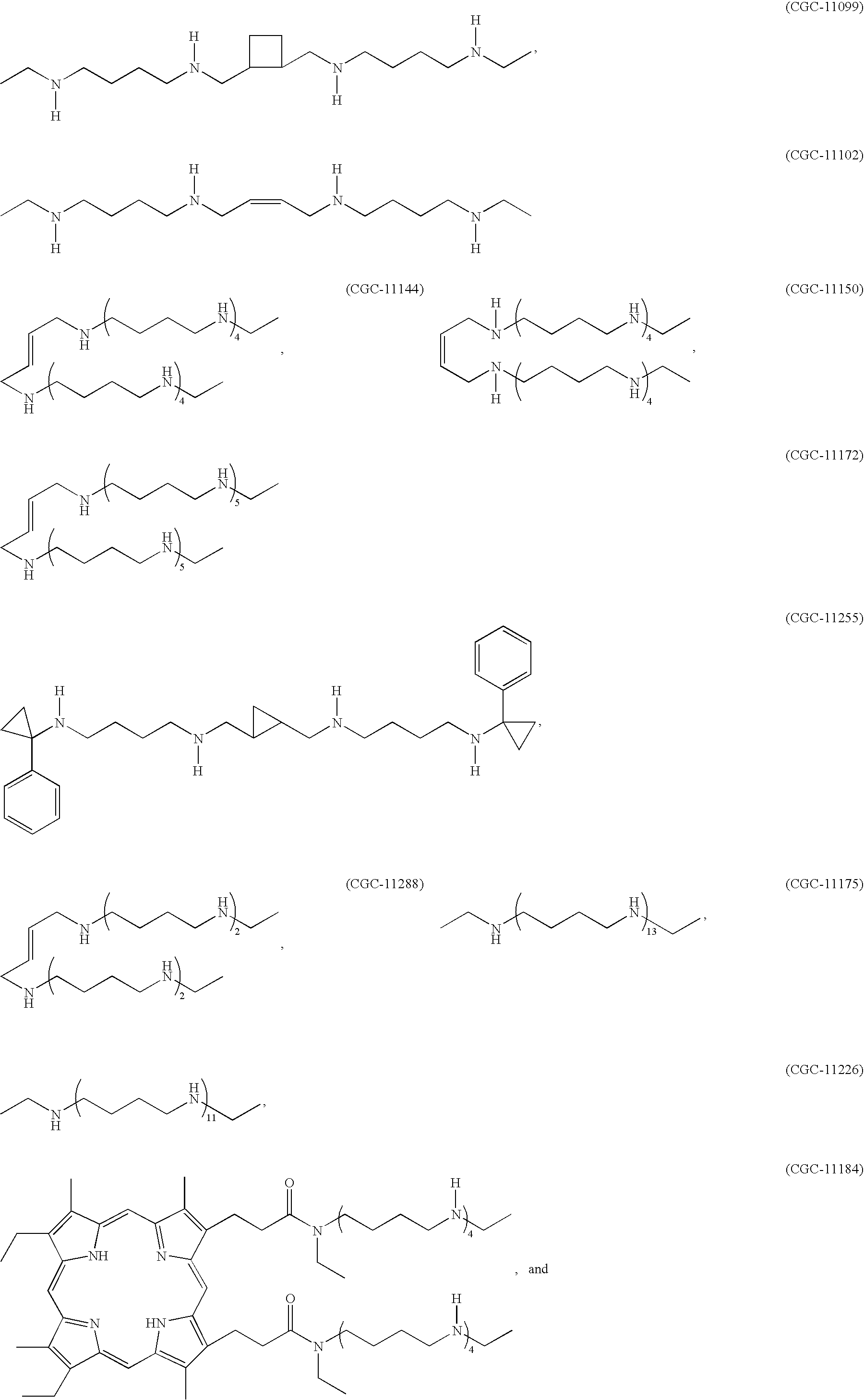 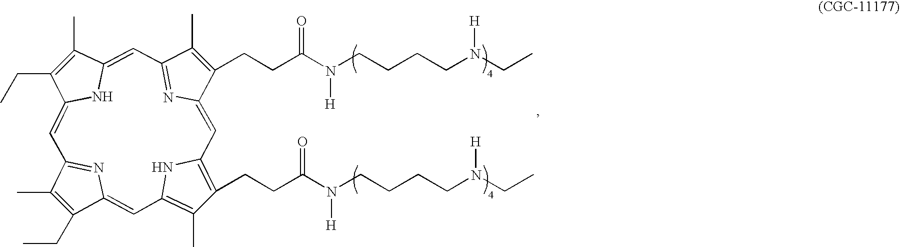
and all salts, solvates, hydrates, and stereoisomers thereof.

4. A method of treating a disease involving cell migration or cell motility, comprising:
administering one or more polyamine analogs to a subject in a therapeutically effective amount to a patient in need thereof.

5. The method of claim 4, wherein the polyamine analog is a conformationally restricted polyamine analog.

6. The method of claim 4, wherein the polyamine analog is selected from the group consisting of: 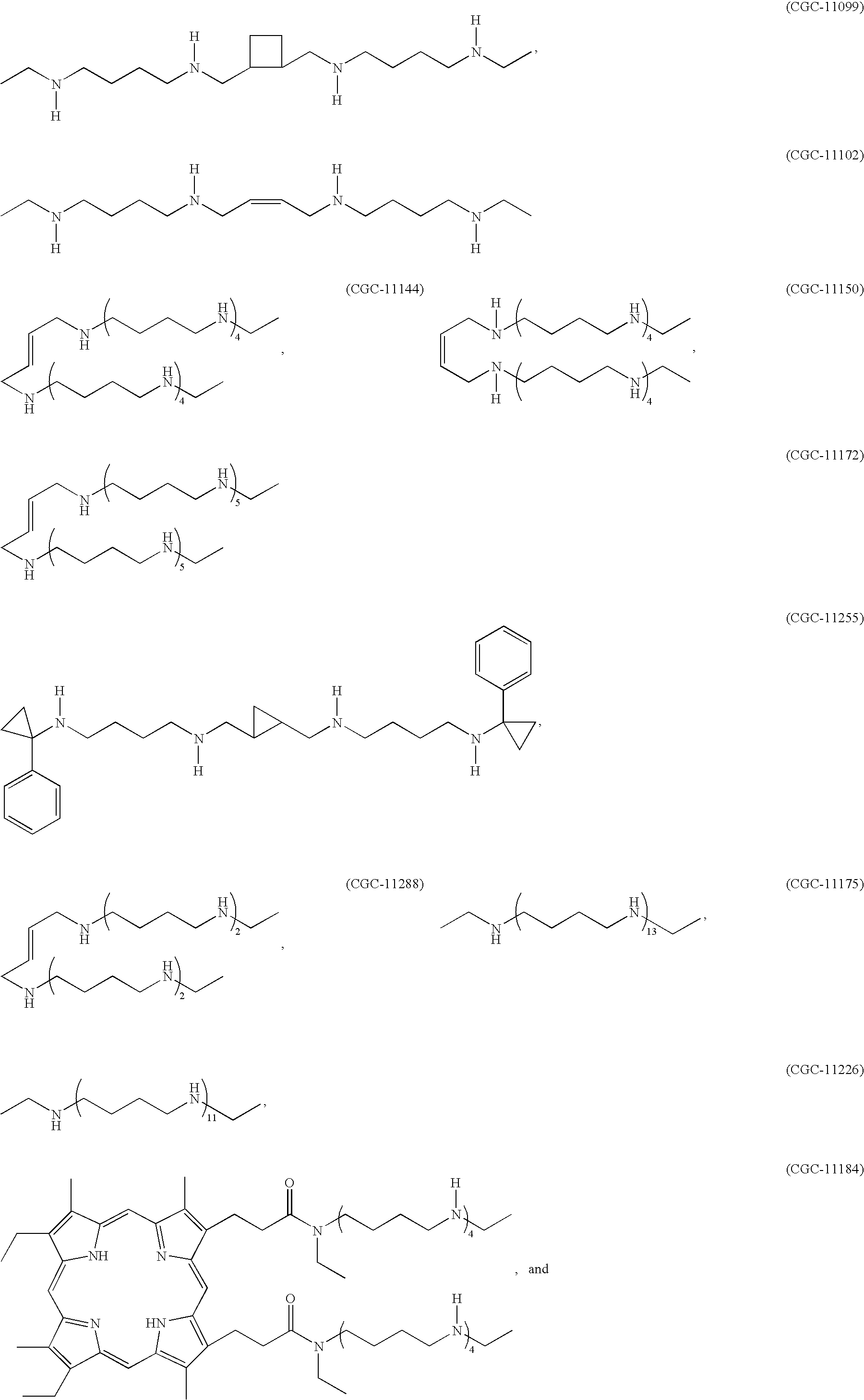 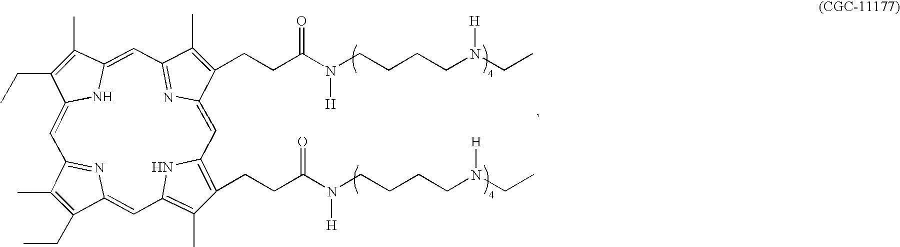
and all salts, solvates, hydrates, and stereoisomers thereof.

7. The method of claim 4, wherein the disease is selected from the group consisting of inflammation, infection, endometriosis, abnormal immune responses, undesired angiogenesis, tumor cell metastasis or invasion, atherosclerosis, vascular graft occlusion, restenosis subsequent to angioplasty, transplant rejection, other complications of transplants, and glomerulonephritis, arthritis, inflammatory responses subsequent to stroke or ischemia, inflammatory bowel diseases, gingivitis and periodontitis, and asthma.

8. A compound of the formula: 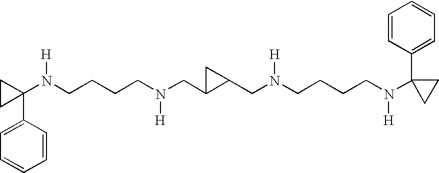
and all salts, hydrates, solvates, and stereoisomers thereof.

9. A compound of claim 8, further comprising a pharmaceutically acceptable carrier.
